(19) United States
(12) Patent Application Publication
(10) Pub. No.: US 2021/0077501 A1
(43) Pub. Date: Mar. 18, 2021

(54) COMPOSITIONS FOR PREVENTING OR TREATING UVEITIS

(71) Applicant: Chong Kun Dang Pharmaceutical Corp., Seoul (KR)

(72) Inventors: Young II Choi, Gyeonggi-do (KR); Nina Ha, Gyeonggi-do (KR); Taek Hwan Shin, Gyeonggi-do (KR)

(21) Appl. No.: 16/970,292

(22) PCT Filed: Feb. 19, 2019
(86) PCT No.: PCT/KR2019/001989

§ 371 Date: Aug. 14, 2020

(30) Foreign Application Priority Data
Feb. 20, 2018 (KR) 1020180020058

(51) Int. Cl. A61K 31/5377; A61P 27/02; A61K 9/00; A61K 31/5375; A61K 31/495; A61K 31/397

(52) U.S. Cl. CPC A61K 31/5377; A61P 27/02; A61K 31/397; A61K 31/5375; A61K 31/495; A61K 9/0048

(57) ABSTRACT

The present invention relates to a pharmaceutical composition for preventing or treating uveitis, containing a compound represented by a formula I, an optical isomer thereof or a pharmaceutically acceptable salt thereof as an effective component, as well as a treatment method using the compound, and use of the compound in the manufacture of a medicament for treating uveitis. The pharmaceutical composition according to the present invention shows an excellent effect of preventing or treating uveitis.

### TECHNICAL FIELD

The present invention relates to a pharmaceutical composition for preventing or treating uveitis, comprising a compound represented by a formula I, an optical isomer thereof or a pharmaceutically acceptable salt thereof as an effective component, as well as a treatment method using the compound, and use of the compound in the manufacture of a medicament for treating uveitis.

### BACKGROUND ART

A uvea is a middle layer inside an outermost layer of an eye ball, which is a cornea and a sclera, wherein the uvea is composed of an iris, a ciliary body and a choroid membrane, and an inflammation developed therein is defined as uveitis. The uvea is susceptible to inflammation because the uvea has abundant blood vessels and many connective tissues, wherein a variety of symptoms thereof occur depending on various causes and a degree of inflammation. As a representative symptom of uveitis, it is known that there are a loss of vision, myodesopsia, pain, bleeding, lacrimation, dazzle, and the like.

Uveitis may be classified into anterior uveitis, intermediate uveitis, posterior uveitis and pan-uveitis depending on a location of its inflammation. Also, uveitis is divided into infectious uveitis caused by viruses or germs, and non-infectious uveitis resulting from an abnormality of the autoimmune system, wherein a majority of such uveitis is caused by immunological factors. So far, however, an accurate pathogenesis of uveitis has not been disclosed yet.

As a representative therapeutic agent for uveitis, steroids or immunosuppressants are used. However, a steroid eye drop lotion often causes serious side effects such as cataract, an increase in intraocular pressure, an increase in inflammation, etc. because the steroid eye drop lotion is used at such a high dosage that its drug may penetrate into a uvea tissue. Also, a long-term use of oral steroids may lead to various side effects such as osteoporosis, osteonecrosis, a suppression of hypothalamic-pituitary-adrenal axis, an increase in infection, abnormalities of metabolism, electrolyte and digestive system, etc. On the other hand, the immunosuppressants, which have been used as an alternative therapy for steroids, are also likely to cause side effects such as bone marrow depression, damages to renal and liver functions, etc. Thus, despite a sufficient treatment with the steroids and immunosuppressants, many problems occur to patients' lives. For example, 5 to 10% of the patients lead to blindness, etc. after all. Thus, there is an urgent need to develop a therapeutic agent for uveitis, which well penetrates into a target tissue with less side effects.

Against these backdrops, the present inventors have made every endeavor to develop the therapeutic agent for uveitis, and thus have identified that a compound according to the present invention may be helpfully used to prevent or treat uveitis and completed the present invention.

### PRIOR ART REFERENCES

### Patent Document

Korea Patent Application Publication No. 2014-0128886

### DISCLOSURE OF INVENTION

### Technical Problem

The objective of the present invention is to provide a pharmaceutical composition for preventing or treating uveitis, comprising a compound represented by a following formula I, an optical isomer thereof or a pharmaceutically acceptable salt thereof as an effective component.

Another objective of the present invention is to provide a method for treating uveitis, wherein the method comprises administering a therapeutically effective amount of the said compound.

Another objective of the present invention is to provide use of the compound in the manufacture of a medicament for treating uveitis.

### Solution to Problem

This will be described in detail as follows. Meanwhile, each description and embodiment form disclosed in the present invention may be applied to other descriptions and embodiment forms thereof, respectively. In other words, all combinations of various elements disclosed in the present invention fall within the scope of the present invention. Also, it cannot be seen that the scope of the present invention is limited to the specific description described below.

The present invention provides a pharmaceutical composition for preventing or treating uveitis, comprising a compound represented by a following formula I, an optical isomer thereof or a pharmaceutically acceptable salt thereof as an effective component:

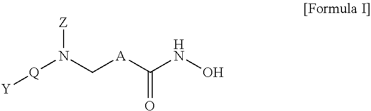

wherein in Formula I,

A is

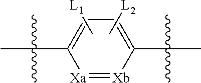

Xa and Xb are each independently CH or N,

L₁ and L₂ are each independently hydrogen, halogen, —CF₃ or —C₁₋₃ straight or branched chain alkyl,

Q is C(═O), S(═O)₂, S(═O) or C(═NH),

Y is selected from a following group:

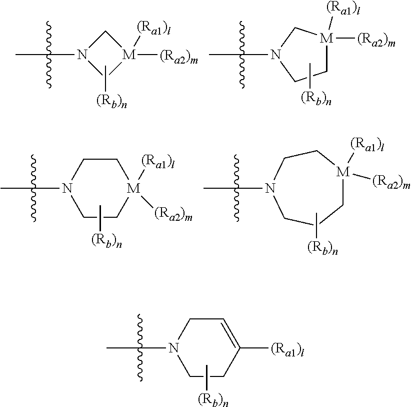

M is C, N, O, S or S(═O)₂, wherein, at this time, in case M is C, l and m are 1; in case M is N, l is 1 and m is 0; and in case M is 0, S or S(═O)₂, l and m are 0,

Rₐ₁ and Rₐ₂ are each independently hydrogen; hydroxy; —C₁₋₄ straight or branched chain alkyl, which is unsubstituted or substituted with at least one halogen; —C₁₋₄ straight or branched chain alcohol; benzhydryl; —C₁₋₄ straight or branched chain alkyl, which is substituted with a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; a saturated or unsaturated 5- to 7-membered heterocyclized compound comprising 1 to 3 heteroatoms of N, O or S as a ring member, wherein at this time, the heterocyclized compound may be unsubstituted or at least one hydrogen may be optionally substituted with OH, OCH₃, CH₃, CH₂CH₃ or halogen; phenyl, wherein it is unsubstituted or at least one hydrogen is substituted with halogen, C₁₋₄ alkoxy, C₁₋₂ alkyl or hydroxy; benzyl, wherein it is unsubstituted or at least one hydrogen is substituted with halogen, C₁₋₄ alkoxy, C₁₋₂ alkyl or hydroxy; —S(═O)₂CH₃; halogen; —C₁₋₆ straight or branched chain alkoxy; —C₂₋₆ alkoxyalkyl; —C(═O)Rₓ, wherein Rₓ is straight or branched chain C₁₋₃ alkyl or C₃₋₁₀ cycloalkyl;

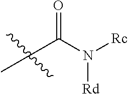

wherein R_{c} and R_{d} are each independently hydrogen, C₁₋₃ straight or branched chain alkyl; and

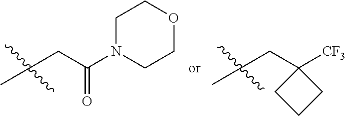 [SMILES: *CC(=O)N1CCOCC1 ; *CC1(C(F)(F)F)CCC1]

n is an integer of 0, 1 or 2,

R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or

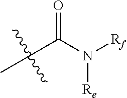

Rₑ and R_{f} are each independently hydrogen or —C₁₋₃ straight or branched chain alkyl,

Z is selected from a following group:

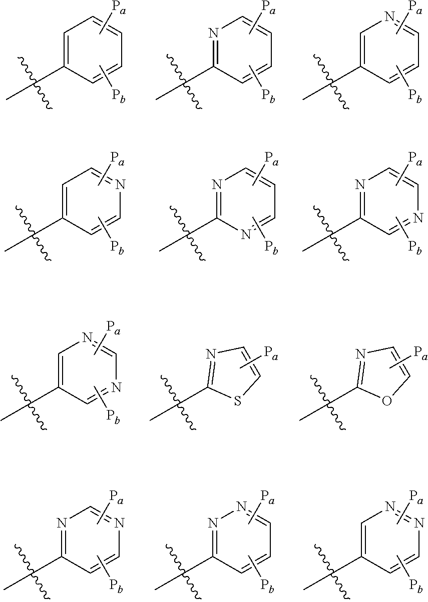

Pₐ and P_{b} are each independently

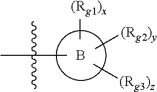

hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol, wherein

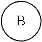

is phenyl, pyridine, pyrimidine, thiazole, indole, indazole, piperazine, quinoline, furan, tetrahydropyridine, piperidine or a ring selected from a following group:

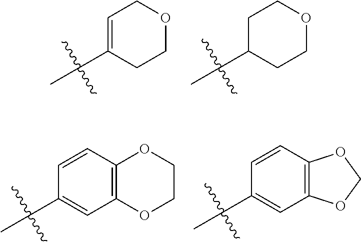 [SMILES: *C1=CCOCC1 ; *C1CCOCC1 ; *c1ccc2c(c1)OCCO2 ; *c1ccc2c(c1)OCO2]

x, y and z are each independently an integer of 0 or 1,

R_{g1}, R_{g2} and R_{g3} are each independently hydrogen; hydroxy; —C₁₋₃ alkyl; —CF₃; —C₁₋₆ straight or branched chain alkoxy; —C₂₋₆ straight or branched chain alkyl alkoxy; —C(═O)CH₃; —C₁₋₄ straight or branched chain hydroxyalkyl; —N(CH₃)₂; halogen; phenyl; —S((═O)₂)CH₃; or selected from a following group:

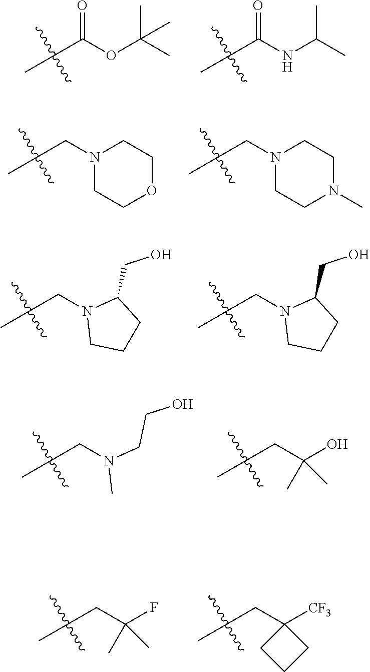

A compound represented by a formula I according to the present invention may be a compound represented by a following formula Ia:

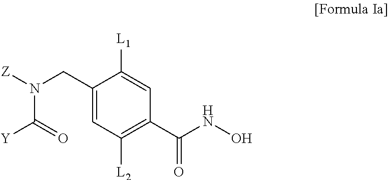

wherein

L₁ and L₂ are each independently hydrogen or halogen,

Y is

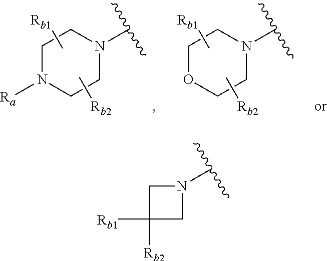

Z is phenyl or pyridinyl, wherein at least one hydrogen of phenyl or pyridinyl may be substituted with halogen, CF₃ or CF₂H.

According to a specific embodiment of the present invention, the compound represented by the formula Ia above is a compound described in a following table 1:

TABLE 1

| Compound | Structure |
|---|---|
| 255 | 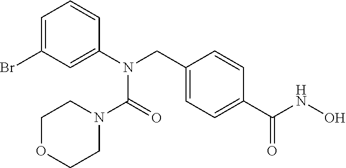 [SMILES: O=C(NO)c1ccc(CN(C(=O)N2CCOCC2)c2cccc(Br)c2)cc1] |
| 280 | 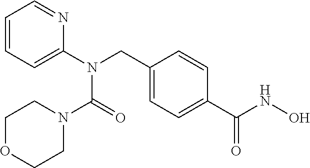 [SMILES: O=C(NO)c1ccc(CN(C(=O)N2CCOCC2)c2ccccn2)cc1] |
| 374 | 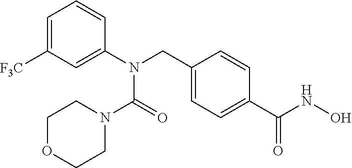 [SMILES: O=C(NO)c1ccc(CN(C(=O)N2CCOCC2)c2cccc(C(F)(F)F)c2)cc1] |
| 416 | 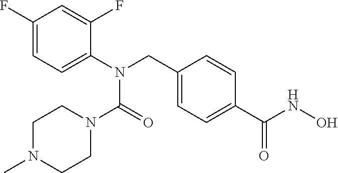 [SMILES: CN1CCN(C(=O)N(Cc2ccc(C(=O)NO)cc2)c2ccc(F)cc2F)CC1] |
| 461 | 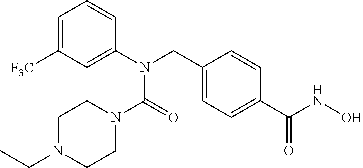 [SMILES: CCN1CCN(C(=O)N(Cc2ccc(C(=O)NO)cc2)c2cccc(C(F)(F)F)c2)CC1] |
| 476 | 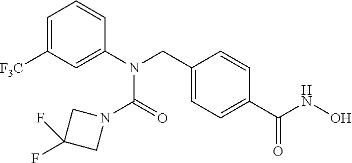 [SMILES: O=C(NO)c1ccc(CN(C(=O)N2CC(F)(F)C2)c2cccc(C(F)(F)F)c2)cc1] |
| 500 | 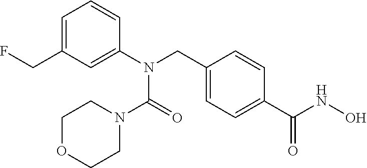 [SMILES: O=C(NO)c1ccc(CN(C(=O)N2CCOCC2)c2cccc(CF)c2)cc1] |
| 530 | 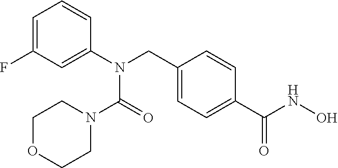 [SMILES: O=C(NO)c1ccc(CN(C(=O)N2CCOCC2)c2cccc(F)c2)cc1] |
| 532 | 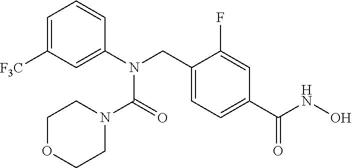 [SMILES: O=C(NO)c1ccc(CN(C(=O)N2CCOCC2)c2cccc(C(F)(F)F)c2)c(F)c1] |

In the present invention, the compound represented by the formula I above may be prepared by means of a method disclosed in Korea Unexamined Patent Application Publication No. 2014-0128886, but is not limited thereto.

In the present invention, a pharmaceutically acceptable salt means a salt conventionally used in an industry of medicine, e.g., an inorganic ion salt prepared from calcium, potassium, sodium, magnesium and the like; an inorganic acid salt prepared from hydrochloric acid, nitric acid, phosphoric acid, bromic acid, iodic acid, perchloric acid, sulfuric acid and the like; an organic acid salt prepared from acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbric acid, carbonic acid, vanillic acid, hydroiodic acid, etc.; a sulphonic acid salt prepared from methanesulfonic acid, ethanesulfonic acid, benzenesulfonic acid, p-toluenesulfonic acid, naphthalenesulfonic acid and the like; amino acid salt prepared from glycine, arginine, lysine, etc.; amine salt prepared from trimethylamine, tri-ethylamine, ammonia, pyridine, picoline, etc.; and the like, but the types of salt meant in the present invention are not limited to those listed salts.

As used herein, the term “uveitis” means an inflammation developed in an inner portion of an eye, particularly the inflammation developed in a middle layer (uvea) of the eye. More particularly, uveitis according to the present invention includes: anterior uveitis, which is the inflammation in an anterior portion of a uvea system, such as the inflammation of an iris (iritis) and the inflammations of the iris and a ciliary body (cyclitis); intermediate uveitis, which is the inflammation in a vitreous body (peripheral uveitis or chronic cyclitis); and posterior uveitis, which is the inflammation in a part of the uvea system behind a crystal lens of the eye, such as the inflammation of a choroid (choroiditis) and the inflammation of the choroid and a retina (chorioretinitis), as well as pan-uveitis, which is uveitis affecting the entire uvea system. Also, according to the present invention, uveitis includes infectious uveitis caused by viruses or germs, and non-infectious uveitis resulting from an autoimmune disease.

In an embodiment of the present invention, it was identified that compounds 255, 280, 374, 416, 461, 476, 500, 530 or 532 represented by a formula Ia had an excellent effect of suppressing an in-vitro production of inflammatory molecules such as TNFα, etc. (FIG. 1), suppressing proliferation of reactive T cells (FIG. 2), and improving a function of regulatory T cells (FIG. 3).

Also, it was identified that a compound 374 according to the present invention has an excellent effect on decreasing a uveitis lesion in an animal model for an induced uveitis disease (FIGS. 4 to 6), inhibiting an infiltration of inflammatory cells (FIGS. 7 to 10), inhibiting an expression of inflammatory cytokines in a spleen and inflammation regions (FIGS. 11 and 12), and decreasing the number of immune cells in a retina and a lymph node (FIGS. 13 to 21).

A pharmaceutical composition according to the present invention may further comprise at least one type of a pharmaceutically acceptable carrier, in addition to the compound represented by the formula I above, the optical isomer thereof or the pharmaceutically acceptable salt thereof, for the purpose of administration. As the pharmaceutically acceptable carrier, saline solution, sterilized water, Ringer's solution, buffered saline, dextrose solution, maltodextrin solution, glycerol, ethanol and a combination of at least one component thereof may be used, wherein other conventional additives such as antioxidant, buffer solution, bacteriostatic agent, etc., may be also added thereto, if needed. Also, the pharmaceutical composition according to the present invention may be formulated into an injectable dosage form such as aqueous solution, suspension, emulsion, etc., pill, capsule, granule or tablet in such a way that diluent, dispersing agent, surfactant, binder and lubricant are further added thereto. Thus, the composition according to the present invention may be a patch, liquid medicine, pill, capsule, granule, tablet, suppository, etc. These preparations may be formulated by means of a conventional method used for formulation in the technical field to which the present invention pertains according to each disease and/or component, or a method disclosed in Remington's Pharmaceutical Science (the latest version), Mack Publishing Company, Easton Pa.

A non-limiting example of a preparation for oral administration using the pharmaceutical composition according to the present invention may be a tablet, troche, lozenge, water soluble suspension, oil suspension, prepared powder, granule, emulsion, hard capsule, soft capsule, syrup, elixir or the like. To formulate the pharmaceutical composition according to the present invention into a preparation for oral administration, a binder such as lactose, saccharose, sorbitol, mannitol, starch, amylopectin, cellulose, gelatin or the like; an excipient such as dicalcium phosphate, etc.; a dis-integrant such as maize starch, sweet potato starch or the like; a lubricant such as magnesium stearate, calcium stearate, sodium stearyl fumarate, polyethylene glycol wax or the like; and so on may be used, and a sweetening agent, flavoring agent, syrup, etc., may be also used. Furthermore, in case of the capsule, a liquid carrier such as fatty oil, etc. may be further used in addition to the above-mentioned materials.

A non-limiting example of a parenteral preparation using the pharmaceutical composition according to the present invention may be an injectable solution, suppository, powder for respiratory inhalation, aerosol preparation for spray, ointment, powder for application, oil, cream, etc. To formulate the pharmaceutical composition according to the present invention into a preparation for parenteral administration, a sterilized aqueous solution, nonaqueous solvent, suspension, emulsion, freeze-dried preparation, external preparation, etc. may be used. As the said nonaqueous solvent and suspension, a vegetable oil such as propylene glycol, polyethylene glycol and olive oil; an injectable ester such as ethyl oleate; and so on may be used, for example, an ophthalmic solution or emulsion, an ophthalmic gel, an ophthalmic ointment or an oily lotion, which contains a composition for eye drop, may be used, but not limited thereto.

The pharmaceutical composition according to the present invention may be orally or parenterally administered, preferably parenterally administered, for example, administered by eye drop or intraperitoneally, but not limited thereto.

If the pharmaceutical composition according to the present invention is used in a form of a composition for eye drop, the said composition for eye drop may be prepared by suspending a compound of Formula I according to the present invention, an optical isomer thereof or a pharmaceutically acceptable salt thereof in sterile aqueous solution, for example, salt water, buffer solution, etc., or by compounding the above-mentioned compositions in a form of soluble powder therein before use.

Other additives, for example, an isotonic agent (e.g. sodium chloride, etc.), a buffer agent (e.g. boric acid, sodium monohydrogen phosphate, sodium dihydrogen phosphate, etc.), a preservative agent (e.g. benzalkonium chloride, benzethonium chloride, chlorobutanol, etc.), a thickening agent (e.g. sugars, for example, lactose, mannitol, maltose, etc.; e.g. hyaluronic acid or salt thereof, for example, sodium hyaluronate, potassium hyaluronate, etc.; e.g. mucopolysaccharides, for example, chondroitin sulfate, etc.; e.g. sodium polyacrylate, a carboxy vinyl polymer, cross-linked polyacrylic acid salt, etc.) may be included in the composition for eye drop.

According to an exemplary embodiment of the present invention, it was identified that the compound represented by Formula I achieves an excellent inhibitory effect on inflammatory regions and systematic inflammations, when being dropped into an eye of a mouse with an induced uveitis disease, and thus showing an effective effect on treating uveitis.

A daily dosage of a compound represented by a formula I according to the present invention, an optical isomer thereof or a pharmaceutically acceptable salt thereof may fall, for example, in a range of about 0.1 to 10,000 mg/kg, in a range of about 1 to 8,000 mg/kg, in a range of about 5 to 6,000 mg/kg, or in a range of about 10 to 4,000 mg/kg, preferably in a range of about 50 to 2,000 mg/kg, but is not limited thereto, wherein such dosage may be also administered once a day or divided into several times a day for administration.

A pharmaceutically effective amount and effective dosage of the pharmaceutical composition according to the present invention may be diversified by means of a method for formulating the pharmaceutical composition into a preparation, an administration mode, an administration time and/or administration route, etc., and may be diversified according to various factors including a type and degree of reactions to be achieved by means of an administration of the pharmaceutical composition, a type of an individual to be administered, age, weight, general health condition, a symptom or severity of disease, gender, diet, excretion, a component of other drug compositions used together at the same time or different times for the corresponding individual, and so on, as well as other similar factors well known in a field of medicine, wherein those skilled in the art may easily determine and prescribe a dosage effective for targeted treatment.

In case of the administration of the pharmaceutical composition according to the present invention, it may be administered once a day or divided into several times a day for administration. The pharmaceutical composition according to the present invention may be administered as an individual therapeutic agent or in combination with other therapeutic agents, and may be also administered sequentially or simultaneously with a conventional therapeutic agent. Considering all the factors above, the pharmaceutical composition according to the present invention may be administered by an amount, which can show the maximum effect with the minimum amount without any side effect, wherein such amount may be easily determined by those skilled in the art to which the present invention pertains.

The pharmaceutical composition according to the present invention may show an excellent effect even when used alone, but may be further used in combination with various methods such as hormone therapy, drug treatment, etc. so as to increase a therapeutic efficiency.

The present invention also provides a method for treating uveitis, wherein the method comprises administering a therapeutically effective amount of the compound represented by the formula I above, the optical isomer thereof or the pharmaceutically acceptable salt thereof into an individual in need.

As used herein, the term “therapeutically effective amount” refers to an amount of the compound represented by the formula I above, the optical isomer thereof or the pharmaceutically acceptable salt thereof which is effective in treating uveitis.

In the treatment method according to the present invention, a suitable total daily dose of the compound represented by the formula I above, the optical isomer thereof or the pharmaceutically acceptable salt thereof may be determined by a doctor in charge within the range of correct medical decision, and may fall, for example, in a range of about 0.1 to 10,000 mg/kg, in a range of about 1 to 8,000 mg/kg, in a range of about 5 to 6,000 mg/kg, or in a range of about 10 to 4,000 mg/kg, and preferably such dose in a range of about 50 to 2,000 mg/kg may be administered once a day or divided into several times a day for administration. However, for the purpose of the present invention, it is preferable that a specific, therapeutically effective amount for a certain patient is differently applied depending on various factors including a type and degree of reactions to be achieved, a specific composition including whether other preparations are used or not in some cases, a patient's age, weight, general health condition, gender and diet, an administration time, an administration route and a secretion rate of the composition, a treatment period, and a drug used together or simultaneously with the specific composition, as well as other similar factors well known in a field of medicine.

The method for treating uveitis according to the present invention comprises not only dealing with the disease itself before expression of its symptoms, but also inhibiting or avoiding such symptoms by administering the compound represented by the formula I above, the optical isomer thereof or the pharmaceutically acceptable salt thereof. In managing the disease, a preventive or therapeutic dose of a certain active component may vary depending on characteristics and severity of the disease or condition, and a route in which the active component is administered. The dose and a frequency thereof may vary depending on an individual patient's age, weight and reactions. A suitable dose and usage may be easily selected by those skilled in the art, naturally considering such factors. Also, the method for treating uveitis according to the present invention may further comprise administering a therapeutically effective dose of an additional active agent, which is helpful in treating the disease, along with the compound represented by the formula I above, the optical isomer thereof or the pharmaceutically acceptable salt thereof wherein the additional active agent may show a synergy effect or an additive effect together with the compound of the formula I above, the optical isomer thereof or the pharmaceutically acceptable salt thereof.

The present invention also provides a use of the compound represented by the formula I above, the optical isomer thereof or the pharmaceutically acceptable salt thereof in the manufacture of a medicament for treating uveitis.

The compound represented by the formula I above, the optical isomer thereof or the pharmaceutically acceptable salt thereof for the manufacture of a medicament may be combined with a pharmaceutically acceptable adjuvant, diluent, carrier, etc., and may be prepared into a composite agent together with other active agents, thus having a synergy action.

The matters mentioned in the inventive pharmaceutical composition, the treatment method and the use are equally applied, if not contradictory to each other.

### Advantageous Effects of Invention

A pharmaceutical composition comprising a compound represented by a formula I according to the present invention, an optical isomer thereof or a pharmaceutically acceptable salt thereof may show an excellent effect of treating uveitis, such that the pharmaceutical composition may be widely used for prevention or treatment of uveitis.

### BRIEF DESCRIPTION OF DRAWINGS

FIG. 1 shows results of identifying an effect of the inventive compound on suppressing TNFα secretion.

FIG. 2 shows results of identifying an effect of the inventive compound on suppressing a proliferation of reactive T cells.

FIG. 3 shows results of identifying an effect of the inventive compound on adjusting a function of regulatory T cells.

FIG. 4 shows a graph of evaluating a clinical grade of uveitis on a retina of an experimental autoimmune uveitis (EAU) mouse (*:p<0.05; **:p<0.01; ***:p<0.001 by Tukey's Multiple Comparison Test).

FIG. 5 shows pictures of identifying a clinical change in the retina of the EAU mouse.

FIG. 6 shows results of hematoxylin & eosin (H&E) staining on the retina of the EAU mouse.

FIG. 7 shows results of immunofluorescent staining on CD3 and B220 in the retina of the EAU mouse.

FIG. 8 shows results of immunofluorescent staining on HDAC6 and CD4 in the retina of the EAU mouse.

FIG. 9 shows results of immunofluorescent staining on HDAC6 and B220 in the retina of the EAU mouse.

FIG. 10 shows results of immunofluorescent staining on HDAC6 and α-tubulin in the retina of the EAU mouse.

FIG. 11 shows results of performing ELISA on IFN-γ and IL-17A in a spleen tissue of the EAU mouse (*:p<0.05; **:p<0.01; ***:p<0.001 by Tukey's Multiple Comparison Test).

FIG. 12 shows results of performing a real-time PCR on HDAC, IL-β, IFN-γ, IL-17 and TNF-α in an ocular tissue of the EAU mouse (V: vehicle; C: CKD4; *:p<0.05; **:p<0.01; ***:p<0.001 by Tukey's Multiple Comparison Test).

FIG. 13 shows results of performing a FACS on IFN-γ(+)/CD4(+) immune cells in a retina tissue of the EAU mouse, to which the compound according to the present invention was administered by eye drop.

FIG. 14 shows results of performing the FACS on IFN-γ(+)/CD4(+) immune cells in the retina tissue of the EAU mouse, to which the compound according to the present invention was intraperitoneally administered.

FIG. 15 shows results of performing the FACS on IFN-γ(+)/CD4(+) immune cells in a lymph node of the EAU mouse, to which the compound according to the present invention was administered by eye drop.

FIG. 16 shows results of performing the FACS on IFN-γ(+)/CD4(+) immune cells in the lymph node of the EAU mouse, to which the compound according to the present invention was intraperitoneally administered.

FIG. 17 shows results of performing the FACS on IL-1β(+) immune cells in the lymph node of the EAU mouse, to which the compound according to the present invention was administered by eye drop.

FIG. 18 shows results of performing the FACS on IL-1β(+) immune cells in the retina tissue of the EAU mouse, to which the compound according to the present invention was administered by eye drop.

FIG. 19 shows results of performing the FACS on CD11b(+) immune cells in the lymph node of the EAU mouse, to which the compound according to the present invention was intraperitoneally administered.

FIG. 20 shows results of performing the FACS on CD19(+) immune cells in the lymph node of the EAU mouse, to which the compound according to the present invention was intraperitoneally administered.

FIG. 21 shows results of performing the FACS on F4/80(+) immune cells in the lymph node of the EAU mouse, to which the compound according to the present invention was intraperitoneally administered.

### MODE FOR THE INVENTION

Hereinafter, the present invention will be described in more detail according to preparation examples and embodiments. However, these preparation examples and embodiments are provided only for the purpose of illustrating the present invention, and thus the present invention is not limited thereto.

Compounds 255, 280, 374, 416, 461, 476, 500, 530 or 532 according to the present invention were prepared by means of a method described in Korea Unexamined Patent Application Publication No. 2014-0128886, and specific preparation examples are described below. A newly named formula in each preparation example is mentioned within a corresponding preparation example only, and the formulas mentioned in at least two preparation examples are independently used in each preparation example.

### Preparation Example 1. Synthesis of Compound 255 {N-(3-bromophenyl)-N-(4-(hydroxycarbamoyl)benzyl)morpholine-4-carboxamide}

### [Step 1] Synthesis of methyl 4-((N-(3-bromophenyl)morpholine-4-carboxamido)methyl)benzoate

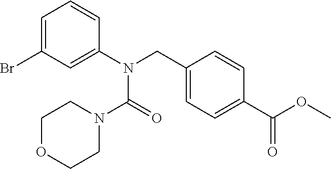 [SMILES: COC(=O)c1ccc(CN(C(=O)N2CCOCC2)c2cccc(Br)c2)cc1]

Methyl 4-(((3-bromophenyl)((4-nitrophenoxy)carbonyl)amino)methyl)benzoate (1.5 g, 3.09 mmol) was dissolved in acetonitrile (50 ml), and then potassium carbonate (1.28 g, 9.3 mmol) and morpholine (0.40 mL, 4.64 mmol) were slowly added thereto. After that, a temperature of a resulting mixture was slowly raised up to 80° C., and then the resulting mixture was stirred for three hours at that temperature. The temperature was cooled down to room temperature, then dimethylformamide (50 ml) was further added thereto, then the temperature was raised up to 80° C. again, and then the resulting mixture was stirred for five hours at that temperature. After a reaction was completed, an organic layer was washed with saturated ammonium chloride aqueous solution three times, then dried by means of sodium sulfate and filtered, and then a filtrate was concentrated under reduced pressure. A concentrate was purified via column chromatography (silicon dioxide; ethyl acetate/hexane=0-50%), such that a title compound (0.45 g, 33.6%) was obtained in a transparent oil form.

### [Step 2] Synthesis of N-(3-bromophenyl)-N-(4-(hydroxycarbamoyl)benzyl)morpholine-4-carboxamide

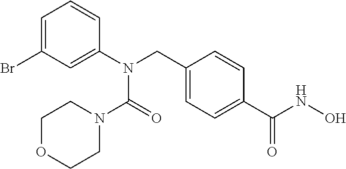 [SMILES: O=C(NO)c1ccc(CN(C(=O)N2CCOCC2)c2cccc(Br)c2)cc1]

Methyl 4-((N-(3-bromophenyl)morpholine-4-carboxamido)methyl)benzoate (0.05 g, 0.12 mmol) was dissolved in methanol (2 ml), and then hydroxylamine hydrochloric acid (0.040 g, 0.58 mmol) was slowly added thereto. After that, potassium hydroxide (0.065 g, 1.15 mmol) was inserted into a resulting mixture, and stirred at room temperature for ten minutes, and then hydroxylamine (50.0 wt % aqueous solution, 0.14 mL, 2.31 mmol) was inserted thereinto. After the resulting mixture was stirred at room temperature for a day, an organic solvent was concentrated under reduced pressure, and then neutralized with the addition of 2N hydrochloric acid. Then, an organic layer was washed with saturated sodium chloride aqueous solution three times, and then dried by means of anhydrous sodium sulfate and filtered. After that, a filtrate was concentrated under reduced pressure, and then a concentrate was purified via column chromatography (silicon dioxide; ethyl acetate/hexane=0-80%), such that a title compound (0.036 g, 72%) was obtained in a white solid form.

¹H NMR (400 MHz, CDCl₃-d₆) δ 7.63 (d, 2H, J=7.8 Hz), 7.27-7.20 (m, 4H), 7.13 (t, 1H, J=7.8 Hz), 6.96 (d, 1H, J=7.1 Hz), 4.83 (s, 2H), 3.49 (brs, 4H), 3.23 (brs, 4H); MS (ESI) m/z 436 (M⁺+H).

### Preparation Example 2. Synthesis of Compound 280 {N-(4-(hydroxycarbamoyl)benzyl)-N-(pyridine-2-yl)morpholine-4-carboxamide}

### [Step 1] Synthesis of methyl 4-((pyridine-2-ylamino)methyl)benzoate

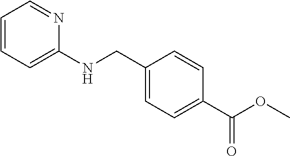 [SMILES: COC(=O)c1ccc(CNc2ccccn2)cc1]

Pyridine-2-amine (0.2 g, 2.13 mmol) was dissolved in methanol (10 mL), and then methyl 4-formylbenzoate (0.35 g, 2.13 mmol) was added thereto. After a resulting mixture was stirred at room temperature for 20 minutes, sodium cyanoborohydride (0.13 g, 2.13 mmol) and acetic acid (0.12 mL. 2.13 mmol) were slowly added thereto, and then stirred at room temperature for five hours. The resulting mixture was washed with saturated sodium chloride aqueous solution three times, then an organic layer was dried by means of sodium sulfate and filtered, and then a filtrate was concentrated under reduced pressure. A concentrate was purified via column chromatography (silicon dioxide; ethyl acetate/hexane=0-30%), such that a title compound (0.10 g, 19%) was obtained in a transparent oil form.

¹H NMR (400 MHz, CDCl₃) δ 8.17 (d, 1H, J=5.8 Hz), 8.06 (d, 2H, J=8.4 Hz), 7.66 (t, 1H, J=7.8 Hz), 7.44 (d, 2H, J=8.0 Hz), 6.76 (t, 1H, J=6.7 Hz), 6.58 (d, 1H, J=8.6 Hz), 4.67 (d, 2H, J=6.0 Hz), 3.92 (s, 3H).

### [Step 2] Synthesis of methyl 4-((((4-nitrophenoxy)carbonyl)(pyridine-2-yl)amino)methyl)benzoate

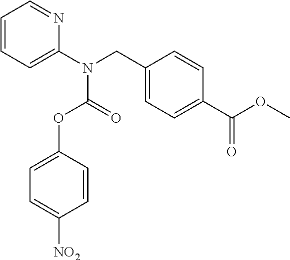 [SMILES: COC(=O)c1ccc(CN(C(=O)Oc2ccc([N+](=O)[O-])cc2)c2ccccn2)cc1]

Methyl 4-((pyridine-2-ylamino)methyl)benzoate (0.040 g, 0.16 mmol) was dissolved in dimethylformamide (3 mL), and then potassium carbonate (0.046 g, 0.33 mmol) was slowly added thereto. After that, 4-nitrophenyl chloroformate (0.037 g, 0.18 mmol) was added to a resulting mixture, then a temperature of the resulting mixture was slowly raised up to 50° C., and then the resulting mixture was stirred for two days at that temperature. After a reaction was completed, an ethyl acetate layer was washed with saturated ammonium chloride aqueous solution three times, then an organic layer was dried by means of sodium sulfate and filtered, and then a filtrate was concentrated under reduced pressure. A concentrate was purified via column chromatography (silicon dioxide; ethyl acetate/hexane=0-50%), such that a title compound (0.048 g, 71%) was obtained in a yellow oil form.

¹H NMR (400 MHz, CDCl₃) δ 8.49-8.48 (m, 1H), 8.24 (dd, 2H, J=7.0, 2.2 Hz), 8.17 (dd, 2H, J=7.2, 2.0 Hz), 8.00 (d, 2H, J=8.4 Hz), 7.78 (t, 1H, J=3.8 Hz), 7.44 (d, 2H, J=8.0 Hz), 6.91 (dd, 2H, J=7.3, 2.1 Hz), 5.39 (brs, 2H), 3.92 (s, 3H); MS (ESI) m/z 408 (M⁺+H).

### [Step 3] Synthesis of Methyl 4-((N-(pyridine-2-yl)morpholine-4-carboxamido)methyl)benzoate

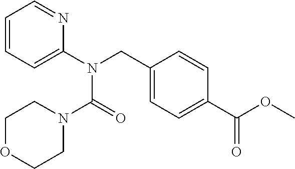 [SMILES: COC(=O)c1ccc(CN(C(=O)N2CCOCC2)c2ccccn2)cc1]

Methyl 4-((((4-nitrophenoxy)carbonyl)(pyridine-2-yl)amino)methyl)benzoate (0.040 g, 0.098 mmol) was dissolved in dimethylformamide (5 ml), and then potassium carbonate (0.040 g, 0.30 mmol) and morpholine (0.013 mL, 0.15 mmol) were slowly added thereto. After that, a temperature of a resulting mixture was slowly raised up to 80° C., and then the resulting mixture was stirred for three hours at that temperature. After a reaction was completed, the resulting mixture was washed with saturated ammonium chloride aqueous solution three times, then an organic layer was dried by means of sodium sulfate and filtered, and then a filtrate was concentrated under reduced pressure. A concentrate was purified via column chromatography (silicon dioxide; ethyl acetate/hexane=0-50%), such that a title compound (0.022 g, 63%) was obtained in a light yellow solid form.

¹H NMR (400 MHz, CDCl₃) δ 8.37-8.35 (m, 1H), 7.95 (d, 2H, J=8.4 Hz), 7.60-7.58 (m, 1H), 7.47 (d, 2H, J=8.4 Hz), 6.94-6.89 (m, 2H), 5.13 (s, 2H), 3.89 (s, 3H), 3.53-3.51 (m, 4H), 3.31-3.29 (m, 4H).

### [Step 4] Synthesis of N-(4-(hydroxycarbamoyl)benzyl)-N-(pyridine-2-yl)morpholine-4-carboxamide

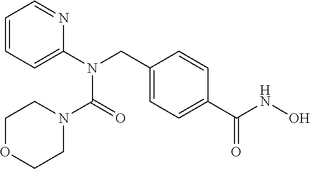 [SMILES: O=C(NO)c1ccc(CN(C(=O)N2CCOCC2)c2ccccn2)cc1]

Methyl 4-((N-(pyridine-2-yl)morpholine-4-carboxamido)methyl)benzoate (0.022 g, 0.062 mmol) was dissolved in MeOH (2 ml), and then hydroxylamine hydrochloric acid (0.022 g, 0.31 mmol) was slowly added thereto. After that, potassium hydroxide (0.035 g, 0.62 mmol) was inserted into a resulting mixture, then stirred at room temperature for ten minutes, and then hydroxylamine (50.0 wt % aqueous solution, 0.082 mL, 1.24 mmol) was inserted thereinto. After the resulting mixture was stirred at room temperature for a day, an organic solvent was concentrated under reduced pressure, then neutralized with the addition of 2N HCl, then washed with saturated sodium chloride aqueous solution three times, and then an organic layer was dried by means of anhydrous sodium sulfate and filtered, such that a title compound (0.007 g, 32%) was obtained in a white solid form.

¹H NMR (400 MHz, MeOD-d 3) δ 8.32 (d, 1H, J=3.6 Hz), 7.72 (t, 1H, J=6.6 Hz), 7.67 (d, 2H, J=8.2 Hz), 7.48 (d, 2H, J=8.2 Hz), 7.08-7.01 (m, 2H), 5.08 (s, 2H), 3.52 (t, 4H, J=4.8 Hz), 3.29 (t, 4H, J=4.8 Hz); MS (ESI) m/z 357 (M⁺+H).

### Preparation Example 3. Synthesis of Compound 374 (CKD4) {N-(4-(hydroxycarbamoyl)benzyl)-N-(3-(trifluoromethyl)phenyl)morpholine-4-carboxamide}

### [Step 1] Synthesis of Methyl 4-((3-(trifluoromethyl)phenylamino)methyl)benzoate

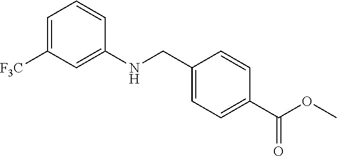 [SMILES: COC(=O)c1ccc(CNc2cccc(C(F)(F)F)c2)cc1]

3-(trifluoromethyl)benzeneamine (0.30 g, 1.84 mmol) and potassium carbonate (0.76 g, 5.53 mmol) were dissolved in dimethylformamide (DMF) (5 mL), and then methyl 4-(bromomethyl)benzoate (0.42 g, 1.84 mmol) was inserted thereinto. A resulting mixture was reacted at room temperature for a day and diluted with ethyl acetate. A reactant was washed with water and saturated sodium chloride aqueous solution, then dried by means of anhydrous magnesium sulfate and filtered, and then concentrated under reduced pressure. A residue was purified via column chromatography (silicon dioxide; ethyl acetate/hexane=20%), such that a title compound (0.37 g, 65%) was obtained.

¹H NMR (400 MHz, DMSO-d₆) δ 7.93 (d, 2H, J=8.3 Hz), 7.49 (d, 2H, J=8.3 Hz), 7.24 (t, 1H, J=7.9 Hz), 6.88-6.78 (m, 4H), 4.42 (d, 2H, J=6.1 Hz), 3.83 (s, 3H), MS (ESI) m/z 310 (M⁺+H).

### [Step 2] Synthesis of methyl 4-((((4-nitrophenoxy)carbonyl)(3-(trifluoromethyl)phenyl)amino)methyl)benzoate

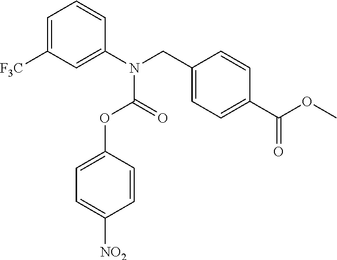 [SMILES: COC(=O)c1ccc(CN(C(=O)Oc2ccc([N+](=O)[O-])cc2)c2cccc(C(F)(F)F)c2)cc1]

Methyl 4-((3-(trifluoromethyl)phenylamino)methyl)benzoate (0.26 g, 0.82 mmol) and 4-nitrophenyl carbonochloridate (0.33 g, 1.65 mmol) were dissolved in acetonitrile (10 mL), and then potassium carbonate (0.34 g, 2.47 mmol) was inserted thereinto. A resulting mixture was reacted at room temperature for a day and diluted with ethyl acetate. A reactant was washed with saturated sodium chloride aqueous solution, then dried by means of anhydrous sodium sulfate and filtered, and then concentrated under reduced pressure. A residue was purified via column chromatography (silicon dioxide; ethyl acetate/hexane=20%), such that a title compound (0.35 g, 89%) was obtained in a colorless oil form.

¹H NMR (400 MHz, CDCl₃) δ 8.20 (d, 2H, J=10.2 Hz), 8.01 (d, 2H, J=7.8 Hz), 7.56-7.46 (m, 3H), 7.35 (d, 3H, J=8.0 Hz), 7.26 (d, 2H, J=8.1 Hz), 5.01 (bs, 2H), 3.90 (s, 3H).

### [Step 3] Synthesis of Methyl 4-((N-(3-(trifluoromethyl)phenyl)morpholine-4-carboxamido)methyl)benzoate

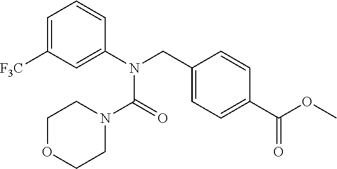 [SMILES: COC(=O)c1ccc(CN(C(=O)N2CCOCC2)c2cccc(C(F)(F)F)c2)cc1]

Methyl 4-((((4-nitrophenoxy)carbonyl)(3-(trifluoromethyl)phenyl)amino)methyl)benzoate (0.29 g, 0.60 mmol) was dissolved in dimethylformamide (10 ml), and then potassium carbonate (0.25 g, 1.81 mmol) and morpholine (0.05 mL, 0.60 mmol) were inserted thereinto. A resulting mixture was reacted at 60° C. for two days, and then diluted with saturated ammonium chloride solution. Extraction was performed by means of ethyl acetate, and then an extract was dried by means of anhydrous sodium sulfate and filtered, and then concentrated under reduced pressure. A residue was purified via column chromatography (silicon dioxide; ethyl acetate/hexane=50%), such that a title compound (0.15 g, 60%) was obtained.

¹H NMR (400 MHz, DMSO-d₆) δ 7.97 (d, 2H, J=8.2 Hz), 7.43-7.32 (m, 5H), 7.20 (d, 1H, J=8.0 Hz), 4.94 (s, 2H), 3.90 (s, 3H), 3.50 (t, 4H, J=4.8 Hz), 3.25 (t, 4H, J=4.8 Hz); MS (ESI) m/z 423 (M⁺+H).

### [Step 4] Synthesis of N-(4-(hydroxycarbamoyl)benzyl)-N-(3-(trifluoromethyl)phenyl)morpholine-4-carboxamide

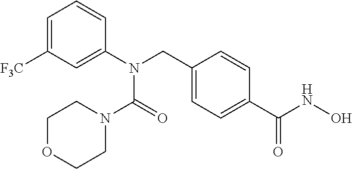 [SMILES: O=C(NO)c1ccc(CN(C(=O)N2CCOCC2)c2cccc(C(F)(F)F)c2)cc1]

Methyl 4-((N-(3-(trifluoromethyl)phenyl)morpholine-4-carboxamido)methyl)benzoate (0.15 g, 0.36 mmol) was dissolved in methanol (5 mL), then hydroxylamine aqueous solution (50 wt %, 1 mL) and potassium hydroxide (0.10 g, 1.81 mmol) were inserted thereinto, and then stirred overnight. After a reaction was completed, methanol was distilled under reduced pressure and removed, and then extraction was performed by means of ethyl acetate and water, and then worked up. A resulting extract was dried by means of anhydrous sodium sulfate and filtered, and then concentrated under reduced pressure. A residue was stirred in diethyl ether, and then a solid product was made, filtered and dried, such that a title compound (0.082 g, 54%) was obtained in a white solid form.

¹H NMR (400 MHz, MeOD-d₃) δ 11.14 (brs, 1H), 8.99 (brs, 1H), 7.85 (d, 2H, J=8.0 Hz), 7.66-7.27 (m, 6H), 4.94 (s, 2H), 3.41 (s, 2H), 3.15 (s, 2H). MS (ESI) m/z 424 (M⁺+H).

### Preparation Example 4. Synthesis of Compound 416 {N-(2,4-difluorophenyl)-N-(4-(hydroxycarbamoyl)benzyl)-4-methylpiperazine-1-carboxamide}

### [Step 1] Synthesis of methyl 4-((N-(2,4-difluorophenyl)-4-methylpiperazine-1-carboxamido)methyl)benzoate

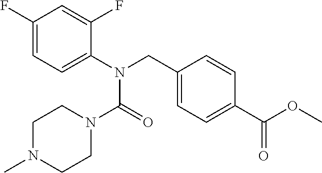 [SMILES: COC(=O)c1ccc(CN(C(=O)N2CCN(C)CC2)c2ccc(F)cc2F)cc1]

Methyl 4-(((2,4-difluorophenyl)((4-nitrophenoxy)carbonyl)amino)methyl)benzoate (0.50 g, 1.13 mmol) and 1-methylpiperazine (0.126 mL, 1.13 mmol) were dissolved in dimethylformamide (10 mL), and then heated and stirred at 60° C. for two days. Dimethylformamide was removed under reduced pressure, then water was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. A residue was purified via column chromatography (silicon dioxide; methanol/dichloromethane=5%) and concentrated, such that a title compound (0.46 g, 101%) was obtained in a yellow oil form.

### [Step 2] Synthesis of N-(2,4-difluorophenyl)-N-(4-(hydroxycarbamoyl)benzyl)-4-methylpiperazine-1-carboxamide

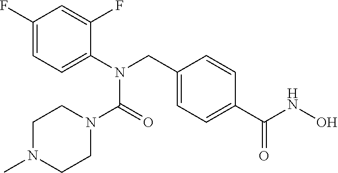 [SMILES: CN1CCN(C(=O)N(Cc2ccc(C(=O)NO)cc2)c2ccc(F)cc2F)CC1]

Methyl 4-((N-(2,4-difluorophenyl)-4-methylpiperazine-1-carboxamido)methyl)benzoate (0.22 g, 0.545 mmol) was dissolved in methanol (20 mL), then hydroxylamine hydrochloric acid (0.189 g, 2.73 mmol) and potassium hydroxide (0.306 g, 5.45 mmol) were added thereto and stirred, then hydroxylamine (50 wt % aqueous solution; 0.701 mL, 10.9 mmol) was added dropwise thereto, and then stirred at room temperature for three hours. After a reaction was completed, methanol was removed under reduced pressure, then water was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. After that, a resulting concentrate was dissolved in dichloromethane, then hexane was added thereto, then a solid was precipitated, filtered and dried, such that a title compound (0.154 g, 70%) was obtained in a yellow solid form.

¹H NMR (400 MHz, MeOD-d₃) δ 7.65 (d, 2H, J=8.2 Hz), 7.40 (d, 2H, J=8.2 Hz), 7.26-7.25 (m, 1H), 7.04-6.96 (m, 2H), 4.79 (s, 2H), 3.25-3.23 (m, 4H), 2.24-2.21 (m, 7H); MS (ESI) m/z 405.1 (M⁺+H).

### Preparation Example 5. Synthesis of Compound 461 {4-ethyl-N-(4-(hydroxycarbamoyl)benzyl)-N-(3-(trifluoromethyl)phenyl)piperazin e-1-carboxamide}

### [Step 1] Synthesis of Methyl 4-((4-ethyl-N-(3-(trifluoromethyl)phenyl)piperazine-1-carboxamido)methyl)benzoate

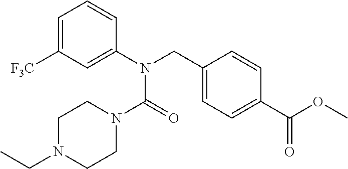 [SMILES: CCN1CCN(C(=O)N(Cc2ccc(C(=O)OC)cc2)c2cccc(C(F)(F)F)c2)CC1]

Methyl 4-((((4-nitrophenoxy)carbonyl)(3-(trifluoromethyl)phenyl)amino)methyl)benzoate (0.346 g, 0.73 mmol) was dissolved in dimethylformamide (10 ml), and then potassium carbonate (0.30 g, 2.19 mmol) and 1-ethylpiperazine (0.09 mL, 0.73 mmol) were inserted thereinto. A resulting mixture was reacted at 60° C. for a day, then diluted with ethyl acetate, and then washed with saturated ammonium chloride solution. The resulting mixture was dried by means of anhydrous magnesium sulfate and filtered, and then concentrated under reduced pressure. A residue was purified via column chromatography (silicon dioxide; ethyl acetate/hexane=50%), such that a title compound (0.15 g, 46%) was obtained.

### [Step 2] Synthesis of 4-ethyl-N-(4-(hydroxycarbamoyl)benzyl)-N-(3-(trifluoromethyl)phenyl)piperazine-1-carboxamide

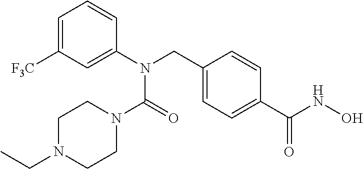 [SMILES: CCN1CCN(C(=O)N(Cc2ccc(C(=O)NO)cc2)c2cccc(C(F)(F)F)c2)CC1]

Methyl 4-((4-ethyl-N-(3-(trifluoromethyl)phenyl)piperazine-1-carboxamido)methyl)benzoate (0.15 g, 0.33 mmol) was dissolved in methanol (10 mL), then hydroxylamine (50 wt % aqueous solution, 0.20 mL) and potassium hydroxide (0.09 g, 1.67 mmol) were added thereinto, and then stirred overnight.

After a reaction was completed, methanol was distilled under reduced pressure and removed, then extraction was performed by means of ethyl acetate and water, and then worked up. A resulting extract was dried by means of anhydrous magnesium sulfate and filtered, and then concentrated under reduced pressure. A residue was stirred in diethyl ether, and then a solid was made, filtered and dried, such that a title compound (0.09 g, 61%) was obtained in a yellow solid form.

¹H NMR (400 MHz, DMSO-d₆) δ 11.1 (brs, 1H), 7.65 (d, 2H, J=8.2 Hz), 7.51 (t, 1H, J=7.9 Hz), 7.41-7.36 (m, 5H), 4.92 (s, 2H), 3.17-3.14 (m, 4H), 2.25, 2.22 (ABq, 2H, J=12.4, 7.2 Hz), 2.18-2.15 (m, 4H), 0.92 (t, 3H, J=7.2 Hz); MS (ESI) m/z 451.1 (M⁺+H).

### Preparation Example 6. Synthesis of Compound 476 {3,3-difluoro-N-(4-(hydroxycarbamoyl)benzyl)-N-(3-(trifluoromethyl)phenyl)azetidine-1-carboxamide}

### [Step 1] Synthesis of Methyl 4-((3,3-difluoro-N-(3-(trifluoromethyl)phenyl)azetidine-1-carboxamido)methyl)benzoate

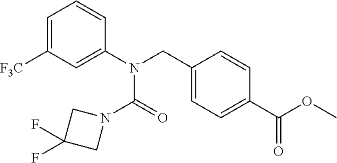 [SMILES: COC(=O)c1ccc(CN(C(=O)N2CC(F)(F)C2)c2cccc(C(F)(F)F)c2)cc1]

Methyl 4-((((4-nitrophenoxy)carbonyl)(3-(trifluoromethyl)phenyl)amino)methyl)benzoate (0.24 g, 0.51 mmol) was dissolved in dimethylformamide (5 ml), and then potassium carbonate (0.21 g, 1.52 mmol) and 3,3-difluoroazetidine hydrochloride (0.13 g, 1.10 mmol) were inserted thereinto. A resulting mixture was reacted at 60° C. for two days, and then diluted with saturated ammonium chloride solution. Extraction was performed by means of ethyl acetate, and then a resulting extract was dried by means of anhydrous sodium sulfate and filtered, and then concentrated under reduced pressure. A residue was purified via column chromatography (silicon dioxide; ethyl acetate/hexane=30%), such that a title compound (0.14 g, 63%) was obtained.

### [Step 2] Synthesis of 3,3-difluoro-N-(4-(hydroxycarbamoyl)benzyl)-N-(3-(trifluoromethyl)phenyl)azetidine-1-carboxamide

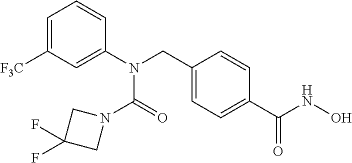 [SMILES: O=C(NO)c1ccc(CN(C(=O)N2CC(F)(F)C2)c2cccc(C(F)(F)F)c2)cc1]

Methyl 4-((3,3-difluoro-N-(3-(trifluoromethyl)phenyl)azetidine-1-carboxamido)methyl)benzoate (0.14 g, 0.32 mmol) was dissolved in methanol (10 mL), then hydroxylamine aqueous solution (50 wt %, 0.2 mL) and potassium hydroxide (0.09 g, 1.60 mmol) were inserted thereinto, and then stirred overnight. After a reaction was completed, methanol was distilled under reduced pressure and removed, and then extraction was performed by means of ethyl acetate and water, and then worked up. A resulting extract was dried by means of anhydrous sodium sulfate and filtered, and then concentrated under reduced pressure. A residue was stirred in diethyl ether, and then a solid product was made, filtered and dried, such that a title compound (0.072 g, 52%) was obtained in a white solid form.

### Preparation Example 7. Synthesis of Compound 500 {N-(3-(fluoromethyl)phenyl)-N-(4-(hydroxycarbamoyl)benzyl)morpholine-4-carboxamide}

### [Step 1] Synthesis of Methyl 4-((N-(3-(fluoromethyl)phenyl)morpholine-4-carboxamido)methyl)benzoate

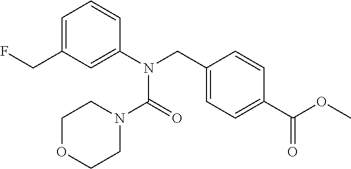 [SMILES: COC(=O)c1ccc(CN(C(=O)N2CCOCC2)c2cccc(CF)c2)cc1]

4-((N-(3-(hydroxymethyl)phenyl)morpholine-4-carboxamido)methyl)benzoic Acid (1.25 g, 3.25 mmol) was dissolved in dichloromethane (20 mL), then diethy-laminosulfur trifluoride (DAST, 0.424 mL, 3.58 mmol) was added thereto at 0° C., then stirred at the same temperature for one hour, then saturated sodium hydrogen carbonate aqueous solution was poured into a resulting reaction mixture, and then extraction was performed by means of dichloromethane. An organic layer was washed with saturated sodium chloride aqueous solution, then dehydrated by means of anhydrous magnesium sulfate, and then concentrated under reduced pressure. A residue was purified via column chromatography (silicon dioxide; ethyl acetate/hexane=30-50%) and concentrated, such that a title compound (0.617 g, 49%) was obtained in a colorless liquid form.

### [Step 2] Synthesis of N-(3-(fluoromethyl)phenyl)-N-(4-(hydroxycarbamoyl)benzyl)morpholine-4-carboxamide

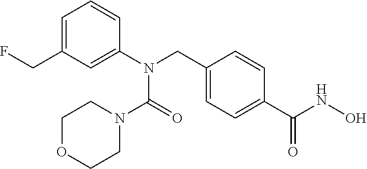 [SMILES: O=C(NO)c1ccc(CN(C(=O)N2CCOCC2)c2cccc(CF)c2)cc1]

Methyl 4-((N-(3-(fluoromethyl)phenyl)morpholine-4-carboxamido)methyl)benzoate (0.100 g, 0.259 mmol) was dissolved in methanol (10 mL), and then hydroxylamine (50.0 wt % aqueous solution, 1.11 mL, 18.1 mmol) was added thereto at room temperature. Then, potassium hydroxide (0.145 g, 2.59 mmol) was added to a resulting mixture and stirred at the same temperature for 30 minutes. After that, solvent was removed from a resulting reaction mixture under reduced pressure, then saturated sodium hydrogen carbonate aqueous solution was poured into a resulting concentrate, and then extraction was performed by means of ethyl acetate. An organic layer was washed with saturated sodium chloride aqueous solution, then dehydrated by means of anhydrous magnesium sulfate, and then concentrated under reduced pressure. Dichloromethane (5 mL) and hexane (30 mL) were inserted into a resulting concentrate and stirred, and then a precipitated solid was filtered and dried, such that a title compound (0.089 g, 89%) was obtained in a white solid form.

¹H NMR (400 MHz, DMSO-d₆) δ 11.12 (brs, 1H), 8.98 (brs, 1H), 7.64 (d, 2H, J=8.3 Hz), 7.36-7.32 (m, 3H), 7.20 (s, 1H), 7.15 (d, 1H, J=7.5 Hz), 7.09 (d, 1H, J=7.4 Hz), 5.36 (d, 2H, J=47.5 Hz), 4.87 (s, 2H), 3.39 (t, 4H, J=4.6 Hz), 3.13 (t, 4H, J=4.6 Hz). MS (ESI) m/z 388 (M⁺+H).

### Preparation Example 8. Synthesis of Compound 530 {N-(3-fluorophenyl)-N-(4-(hydroxycarbamoyl)benzyl)morpholine-4-carboxamide}

### [Step 1] Synthesis of methyl 4-((3-fluorophenylamino)methyl)benzoate

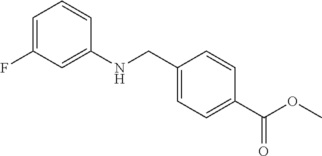 [SMILES: COC(=O)c1ccc(CNc2cccc(F)c2)cc1]

Methyl 4-formylbenzoate (1.47 g, 8.99 mmol) was dissolved in methanol (50 mL), and then 3-fluorobenzeneamine (1.0 g, 8.99 mmol) was inserted thereinto. A resulting mixture was reacted at room temperature for three hours, and then sodium cyanoborohydride (NaCNBH3) (0.56 g, 8.99 mmol) and acetic acid (1.03 mL, 17.99 mmol) were inserted thereinto. After a reactant was reacted at room temperature for a day, reactant solvent was put under reduced pressure and removed, then saturated sodium hydrogen carbonate aqueous solution was poured thereinto, and then extraction was performed by means of ethyl acetate. An organic layer was dehydrated by means of anhydrous magnesium sulfate, and then concentrated under reduced pressure. A residue was purified via column chromatography (silicon dioxide; ethyl acetate/hexane=20%), such that a title compound (1.84 g, 79%) was obtained.

### [Step 2] Synthesis of Methyl 4-(((3-fluorophenyl)((4-nitrophenoxy)carbonyl)amino)methyl)benzoate

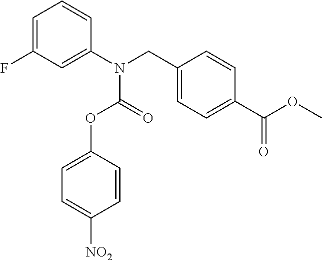 [SMILES: COC(=O)c1ccc(CN(C(=O)Oc2ccc([N+](=O)[O-])cc2)c2cccc(F)c2)cc1]

Methyl 4-((3-fluorophenylamino)methyl)benzoate (2.7 g, 10.4 mmol) and 4-nitrophenyl chloroformate (4.20 g, 20.8 mmol) were dissolved in acetonitrile (100 mL), and then potassium carbonate (4.32 g, 31.2 mmol) was inserted thereinto. A resulting mixture was reacted at room temperature for a day and diluted with ethyl acetate. A reactant was washed with saturated sodium chloride aqueous solution, then dried by means of anhydrous sodium sulfate and filtered, and then concentrated under reduced pressure. A residue was purified via column chromatography (silicon dioxide; ethyl acetate/hexane=20%), such that a title compound (2.65 g, 60%) was obtained in a colorless oil form.

### [Step 3] Synthesis of Methyl 4-((N-(3-fluorophenyl)morpholine-4-carboxamido)methyl)benzoate

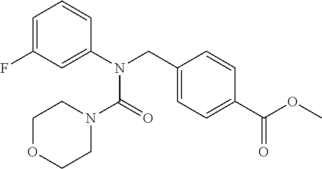 [SMILES: COC(=O)c1ccc(CN(C(=O)N2CCOCC2)c2cccc(F)c2)cc1]

Methyl 4-(((3-fluorophenyl)((4-nitrophenoxy)carbonyl)amino)methyl)benzoate (0.32 g, 0.75 mmol) was dissolved in dimethylformamide (5 ml), and then potassium carbonate (0.31 g, 2.24 mmol) and morpholine (0.13 mL, 1.49 mmol) were inserted thereinto. A resulting mixture was reacted at 60° C. for a day, and then diluted with saturated ammonium chloride solution. Extraction was performed by means of ethyl acetate, and then a resulting extract was dried by means of anhydrous sodium sulfate and filtered, and then concentrated under reduced pressure. A residue was purified via column chromatography (silicon dioxide; ethyl acetate/hexane=30%), such that a title compound (0.13 g, 45%) was obtained.

### [Step 4] Synthesis of N-(3-fluorophenyl)-N-(4-(hydroxycarbamoyl)benzyl)morpholine-4-carboxamide

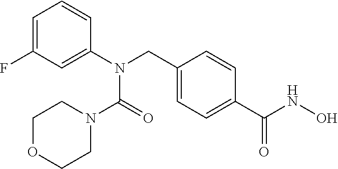 [SMILES: O=C(NO)c1ccc(CN(C(=O)N2CCOCC2)c2cccc(F)c2)cc1]

Methyl 4-((N-(3-fluorophenyl)morpholine-4-carboxamido)methyl)benzoate (0.108 g, 0.290 mmol) was dissolved in methanol (10 mL), and then hydroxylamine (50.0 wt % aqueous solution, 1.19 mL, 19.4 mmol) were added thereto at room temperature. Then, potassium hydroxide (0.156 g, 2.78 mmol) was added to a resulting mixture and stirred at the same temperature for 16 hours. After that, solvent was removed from a resulting reaction mixture under reduced pressure, then saturated sodium hydrogen carbonate aqueous solution was poured into a resulting concentrate, and then extraction was performed by means of ethyl acetate. An organic layer was washed with saturated sodium chloride aqueous solution, then dehydrated by means of anhydrous magnesium sulfate, and then concentrated under reduced pressure. A precipitated solid was filtered and dried, such that a title compound (0.062 g, 57%) was obtained in a white solid form.

¹H NMR (400 MHz, DMSO-d₆) δ 11.14 (brs, 1H), 8.99 (brs, 1H), 7.65 (d, 2H, J=7.0 Hz), 7.38-7.30 (m, 3H), 7.05-6.85 (m, 3H), 4.89 (s, 1H), 3.44-3.42 (m, 4H), 3.18-3.15 (m, 4H), 2.08 (s, 3H). MS (ESI) m/z 374 (M⁺+H).

### Preparation Example 9. Synthesis of Compound 532 {N-(2-fluoro-4-(hydroxycarbamoyl)benzyl)-N-(3-(trifluoromethyl)phenyl)morpholine-4-carboxamide}

### [Step 1] Synthesis of 3-fluoro-4-(((3-(trifluoromethyl)phenyl)amino)methyl)benzonitrile

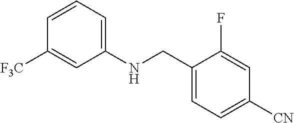 [SMILES: N#Cc1ccc(CNc2cccc(C(F)(F)F)c2)c(F)c1]

3-(trifluoromethyl)aniline (0.998 mL, 8.068 mmol) was dissolved in acetonitrile (60 mL), and then 4-(bromomethyl)-3-fluorobenzonitrile (2.072 g, 9.682 mmol) and DIPEA (2.143 mL, 12.102 mmol) were added thereto at room temperature, and then stirred at the same temperature for a day. After that, saturated sodium hydrogen carbonate aqueous solution was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. A resulting concentrate was purified via column chromatography (silicon dioxide; ethyl acetate/hexane=5-20%) and concentrated, such that a title compound (2.380 g, 64.4%) was obtained in a yellow liquid form.

### [Step 2] Synthesis of 3-fluoro-4-(((3-(trifluoromethyl)phenyl)amino)methyl)benzoic Acid

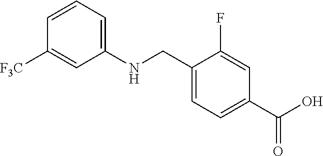 [SMILES: O=C(O)c1ccc(CNc2cccc(C(F)(F)F)c2)c(F)c1]

3-fluoro-4-(((3-(trifluoromethyl)phenyl)amino)methyl)benzonitrile (2.310 g, 7.850 mmol) and lithium hydroxide (3.294 g, 78.505 mmol) were mixed in methanol (40 mL)/H2O (20 mL), then a resulting reaction mixture was heated under reflux for 16 hours, then cooled down to room temperature, and then the resulting reaction mixture was concentrated under reduced pressure. 2M hydrochloric acid aqueous solution was inserted into the resulting mixture to reach pH=1, and then a precipitated solid was filtered and dried, such that a title compound (1.700 g, 69.1%) was obtained in a white solid form.

### [Step 3] Synthesis of Methyl 3-fluoro-4-(((3-(trifluoromethyl)phenyl)amino)methyl)benzoate

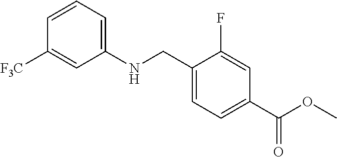 [SMILES: COC(=O)c1ccc(CNc2cccc(C(F)(F)F)c2)c(F)c1]

3-fluoro-4-(((3-(trifluoromethyl)phenyl)amino)methyl)benzoic acid (1.700 g, 5.427 mmol), methanol (4.402 mL, 108.540 mmol), EDC (2.081 g, 10.854 mmol), HOBt (1.467 g, 10.854 mmol) and DIPEA (2.883 mL, 16.281 mmol) were dissolved in tetrahydrofuran (50 mL) at room temperature, then a resulting reaction solution was stirred at the same temperature for 16 hours, then saturated sodium hydrogen carbonate aqueous solution was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. A concentrate was purified via column chromatography (silicon dioxide; ethyl acetate/hexane=10-40%) and concentrated, such that a title compound (1.500 g, 84.5%) was obtained in a colorless liquid form.

### [Step 4] Synthesis of Methyl 3-fluoro-4-((((4-nitrophenoxy)carbonyl)(3-(trifluoromethyl)phenyl)amino)methyl)benzoate

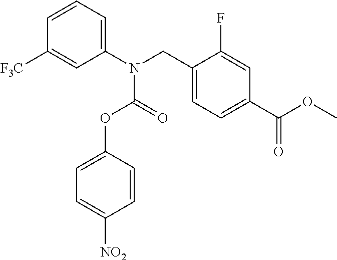 [SMILES: COC(=O)c1ccc(CN(C(=O)Oc2ccc([N+](=O)[O-])cc2)c2cccc(C(F)(F)F)c2)c(F)c1]

Methyl 3-fluoro-4-(((3-(trifluoromethyl)phenyl)amino)methyl)benzoate (1.500 g, 4.583 mmol), 4-nitrophenyl carbonochloridate (1.848 g, 9.167 mmol) and potassium carbonate (1.900 g, 13.750 mmol) were dissolved in acetonitrile (80 mL) at room temperature, then a resulting reaction solution was stirred at the same temperature for 16 hours, then saturated sodium hydrogen carbonate aqueous solution was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. A resulting concentrate was purified via column chromatography (silicon dioxide; ethyl acetate/hexane=10-40%) and concentrated, such that a title compound (0.927 g, 41.1%) was obtained in a colorless liquid form.

### [Step 5] Synthesis of Methyl 3-fluoro-4-((N-(3-(trifluoromethyl)phenyl)morpholine-4-carboxamido)methyl)benzoate

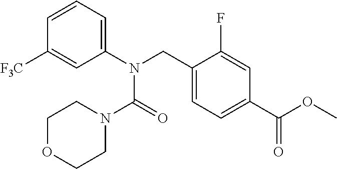 [SMILES: COC(=O)c1ccc(CN(C(=O)N2CCOCC2)c2cccc(C(F)(F)F)c2)c(F)c1]

Methyl 3-fluoro-4-((((4-nitrophenoxy)carbonyl)(3-(trifluoromethyl)phenyl)amino)methyl)benz oate (0.129 g, 0.262 mmol), morpholine (0.046 mL, 0.524 mmol) and potassium carbonate (0.109 g, 0.786 mmol) were dissolved in N,N-dimethylformamide (5 mL) at 60° C., then a resulting reaction solution was stirred at the same temperature for two days, then saturated sodium hydrogen carbonate aqueous solution was poured into a resulting reaction mixture, and then extraction was performed by means of ethyl acetate. An organic layer was washed with saturated sodium chloride aqueous solution, then dehydrated by means of anhydrous magnesium sulfate, and then concentrated under reduced pressure. A resulting concentrate was purified via column chromatography (silicon dioxide; ethyl acetate/hexane=30-60%) and concentrated, such that a title compound (0.094 g, 81.5%) was obtained in a colorless liquid form.

### [Step 6] Synthesis of N-(2-fluoro-4-(hydroxycarbamoyl)benzyl)-N-(3-(trifluoromethyl)phenyl)morpholine-4-carboxamide

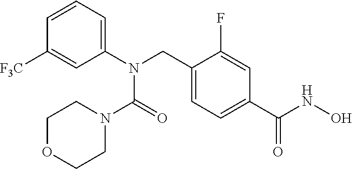 [SMILES: O=C(NO)c1ccc(CN(C(=O)N2CCOCC2)c2cccc(C(F)(F)F)c2)c(F)c1]

Methyl 3-fluoro-4-((N-(3-(trifluoromethyl)phenyl)morpholine-4-carboxamido)methyl)benzoat e (0.094 g, 0.213 mmol) and hydroxylamine (50.0 wt % aqueous solution, 0.071 g, 2.134 mmol) were dissolved in methanol (5 mL), then potassium hydroxide (0.060 g, 1.067 mmol) was added thereto at room temperature, then stirred at the same temperature for two hours, and then a resulting reaction mixture was concentrated under reduced pressure. Diethyl ether (10 mL) was inserted into a resulting concentrate and stirred, and then a precipitated solid was filtered and dried, such that a compound 532 (0.068 g, 72.2%) was obtained in a bright yellow solid form.

¹H NMR (400 MHz, DMSO-d₆) δ 11.2 (brs, 1H), 9.13 (brs, 1H), 7.57-7.42 (m, 7H), 4.94 (s, 2H), 3.44-3.34 (m, 4H), 3.18-3.12 (m, 4H); MS (ESI) m/z 442.1 (M⁺+H).

### Example 1. Identification of Suppressive Effect on TNFα Secretion in Immune Cell Lines (In Vitro)

To identify the efficacy of the inventive compounds on suppressing a TNFα secretion in immune responses, suppression of TNFα production, which was achieved by a treatment with compounds 374, 461, 500, 530 and 532 according to the present invention in LPS-stimulated human monocyte cell lines (THP-1), was quantified by means of an enzyme immuno-assay (ELISA).

Specifically, the THP-1 cell lines (ATCC) were cultured in an RPMI-1640 medium comprising 10% FBS. The cell lines were divided into a 24 well plate at a ratio of 1×10⁵ cells per well, then treated with 100 ng/mL PMA (phorbol 12-myristate 13-acetate) for 24 hours, and then differentiated into macrophage. Then, the culture medium was replaced with a new one, then treated with a test drug for 24 hours, and then treated again with 10 ng/mL LPS (E.Coli, O55:B5) for four hours for stimulation. After that, supernatant was taken and used to measure the amount of TNFα secreted from the cells by means of a Human TNFα Instant ELISA kit (eBioscience, BMS223INST) according to a protocol provided by a manufacturer.

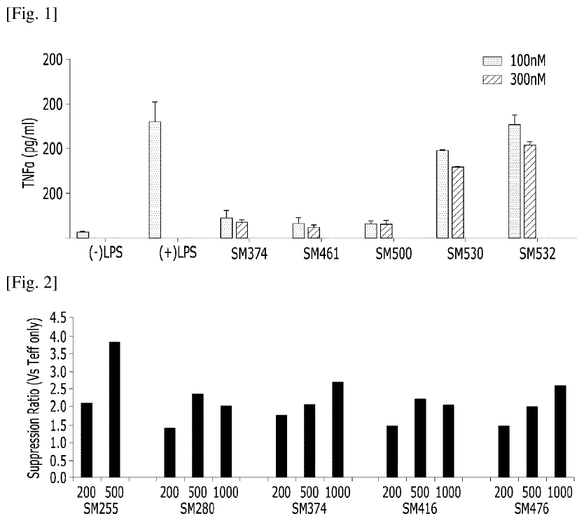

As a result, it was shown that a level of TNFα secretion was decreased in all experimental groups in comparison with a control group, in which an inflammatory response was induced by means of the LPS. In FIG. 1, the compounds denoted as SM374, SM461, SM500, SM530 and SM532 are compounds 374, 461, 500, 530 and 532, respectively. In particular, in case of the compounds 374, 461 and 500, the level of TNFα secretion was remarkably decreased down to a level at which no inflammatory response was induced by means of the LPS at both 100 nM and 300 nM concentrations. Also, in case of increasing a concentration of compound treatment from 100 nM to 300 nM in the compounds 530 and 532, the level of TNFα secretion was drastically decreased (FIG. 1).

The experimental results above show that the compound according to the present invention very effectively suppresses the secretion of TNFα, i.e., an inflammatory response factor, which is representatively increased in uveitis, thus effectively suppressing the inflammatory responses caused in uveitis.

### Example 2. Identification of Suppressive Effect on Reactive T Cells Proliferation (In Vitro)

To identify the efficacy of the inventive compounds on suppressing the proliferation of reactive T cells in immune responses, compounds 255, 280, 374, 416 and 476 according to the present invention were cultured together with reactive T cells and regulatory T cells in LPS-stimulated human monocyte cell lines (THP-1), and then the suppressive efficacy of regulatory T cells was measured.

Specifically, a six-week old C57BL6 male mice were supplied from Central Lab Animal Inc., then acclimated for one week, and then used in an experiment. A spleen was isolated from the mouse, and then treated with collagenase D (Roche, 11088866001), such that splenocytes were isolated therefrom. T_{reg}(CD4+CD25−) and T_{eff} (CD4+CD25+) were isolated by means of a CD4+CD25+ regulatory T cell isolation kit (Miltenyi Biotec, 130-091-041) according to a protocol provided by a manufacturer. T_{eff} cells were cultured at 37° C. for ten minutes by means of eFluor®670 (Cell proliferation Dye eFluor®670, eBioscience), such that cell membranes were stained. T_{eff} and T_{reg} were divided into a 96 well plate at a ratio of 2:1, and then T cells were activated for three days by means of an anti-CD3E and anti-CD28 mAb magnetic bead (T cell activation/expansion kit, Miltenyi Biotec, 130-093627), such that Treg suppression assay was performed. A test drug was simultaneously treated for three days, during which the assay was performed. The divided amount of eFluor®670 labeled on the T_{eff} cell membranes was measured, such that a degree of proliferation of T cells was evaluated accordingly. An eFluor®670-dilution plot was measured by means of a flow cytometer (FACS LSR Fortessa, BD bioscience). The suppressive ability on T cells proliferation was calculated by means of a following equation.

$$Relative suppression = \frac{\% Division ( {T}_{eff} only ) - \% Division ( Drug )}{\% Division ( {T}_{eff} only ) - \% Division ( vehicle )}$$

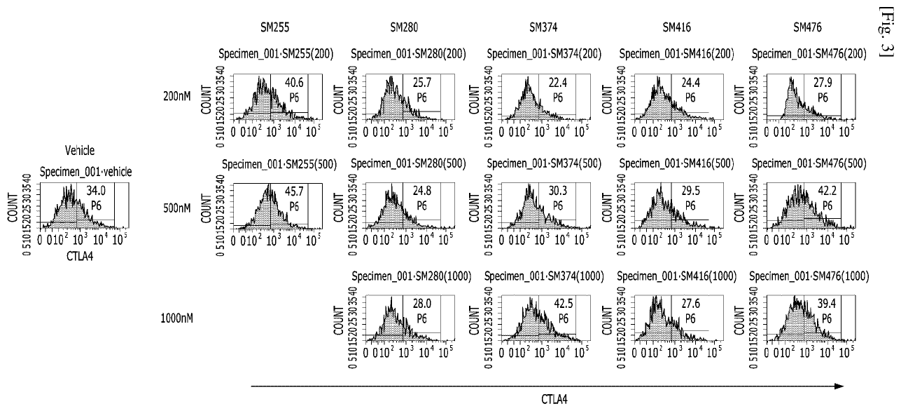

As a result, it was shown that the proliferation of the reactive T cells was suppressed in all the experimental groups. In FIG. 2, the compounds denoted as SM255, SM280, SM374, SM416, SM476 are compounds 255, 280, 374, 416, 476, respectively. The compounds according to the present invention used in the experiment showed a suppression ratio of reactive T cells proliferation, which exceeded maximum two-fold, when treated at 200 nM, and also showed a remarkable effect of suppressing T cells proliferation, which reached maximum four-fold, when treated at 500 nM (FIG. 2).

The experimental results above show that the compounds according to the present invention effectively suppresses the differentiation of the reactive T cells, which were excessively activated in uveitis.

### Example 3. Identification of Regulatory Effect of Regulatory T Cells Function (In Vitro)

To identify if the compounds according to the present invention regulate a function of regulatory T cells in immune responses, compounds 255, 280, 374, 416 and 476 were treated, and then an expression level of an immune checkpoint receptor CTLA4 (cytotoxic T-lymphocyte-associated protein 4) in the regulatory T cells was measured by means of flow cytometry.

Specifically, a six-week old C57BL6 male mice were supplied from Central Lab Animal Inc., then acclimated for one week, and then used in an experiment. A spleen was isolated from the mouse, and then treated with collagenase D (Roche, 11088866001), such that splenocytes were isolated therefrom. CD4+CD25-T cells were isolated by means of a CD4+CD25+ regulatory T cell isolation kit (Miltenyi Biotec, 130-091-041), and then CD4+CD25-T cells (at a ratio of 5×10⁵ cells/well) were treated with an anti-CD3E/anti-CD28 mAb bead (T cell activation/expansion kit, Miltenyi Biotec, 130-093627) and a mouse recombinant TGF-β2 for six days, such that they were differentiated into iT_{reg}. A test drug was simultaneously treated for six days, during which the cells were differentiated into iTreg. After that, the cells were incubated by means of anti-CD4/anti-CD25 mAb (eBioscience, 25-0042-82, 17-0251-82) at 4° C. for 20 minutes, and then labeling was performed. For intracytoplasmic staining, permeabilization was performed by means of Fix/permeabilization buffer (eBioscience, 00-5523-00), then labeling was performed by means of anti-FOXP3-Alexafluor488 (eBioscience, 53-5773-82) and anti-CTLA4-PE (eBioscience, 12-1522-82), and then flow cytometry was performed by means of FACS LSR Fortessa (BD bioscience).

As a result, it was identified that a level of CTLA4 expression in the T cells was increased after treated with the compounds according to the present invention. In FIG. 3, the compounds denoted as SM255, SM280, SM374, SM416, SM476 are compounds 255, 280, 374, 416, 476, respectively. In particular, in case of compounds 255, 374 and 476, it was shown that the CTLA4 expression in 40% or more of the T cells was increased at a concentration of 500 nM or more. The compound 255 showed severe cytotoxicity when treated at 1000 nM, such that data analysis was not performed (FIG. 3).

The experimental results above show that the compounds according to the present invention improve a function of the regulatory T cells, such that an excessive activity of the reactive T cells caused in uveitis might be effectively regulated.

### Preparation Example 10. Establishment of an Animal Model for Induced Experimental Autoimmune Uveitis (EAU)

An animal model for induced experimental autoimmune uveitis (EAU) may be considered as a clinical model for uveitis. As the said EAU animal, a mouse immunized with interphotoreceptor retinoid-binding protein (IRBP) was used.

Particularly, 6 to 8-week old C57BL/6 mice (disease state: severe) were respectively injected with 0.1 ml of a mixture via both sides of a mouse footpad, using a 23 G needle, wherein the mixture was formulated by combining 250 μg of IRBP human peptide (651-670) and 250 μg of Mycobacterium tuberculosis with Complete Freund's adjuvant (CFA) at a ratio of 1:1. On the same day (Day 0) and two days after (Day 2), the mice were intraperitoneally administered with an injection of 0.5 μg/0.2 m of pertussis toxin (PTX) as an adjuvant. The said mice were divided into groups as shown in a following Table 2 depending on whether a compound (CKD4) according to the present invention is applied or not and an administration route thereof (eye drop or intraperitoneal (i.p.) administration).

TABLE 2

| | | | | | Number |
|---|---|---|---|---|---|
| | | | | | of |
| | | | | | animals |
| | | | Admin- | Number of | (number |
| | Group | Con- | istration | admin- | of eye |
| Group | name | centration | route | istrations | balls) |
| 1 | Normal | — | — | — | 4(8) |
| | (CTL) | | | | |
| 2 | EAU | — | — | — | 4(8) |
| 3 | EAU | — | Eye drop | Twice/day | 4(8) |
| | Vehicle | | i.p. | Once/day | |
| | (V) | | | | |
| 4 | EAU + | 0.3% | Eye drop | Twice/day | 4(8) |
| | CKD₄ | 10 or 30 | i.p. | Once/day | |
| | | mg/kg | | | |

A group administered with the compound (CKD4) according to the present invention was administered 0.3% of the CKD4 compound dissolved in a water phase part by eye drop twice a day from Day 11 (an eye drop administration group), or intraperitoneally administered with 10 or 30 mg/kg thereof once a day (an i.p. administration group). On Day 21, a clinical grading was performed, then mice were sacrificed, and then eye balls and target organs thereof were removed therefrom, such that a follow-up experiment was performed.

### Example 4. Identification of an Improvement Effect on Clinical Grading in the EAU Mouse

On Day 21, clinical indicators on uveitis were observed after taking a picture of the periphery of optic nerves of mice with a fundoscopic camera. The severity of uveitis was graded as Grade 0 (normal), Grade 0.5 (very mild), Grade 1 (mild), Grade 2 (moderate), Grade 3 (severe), or Grade 4 (very severe), as described in the document (Rodent Immunology Model Book; Chapter 22; FIG. 2).

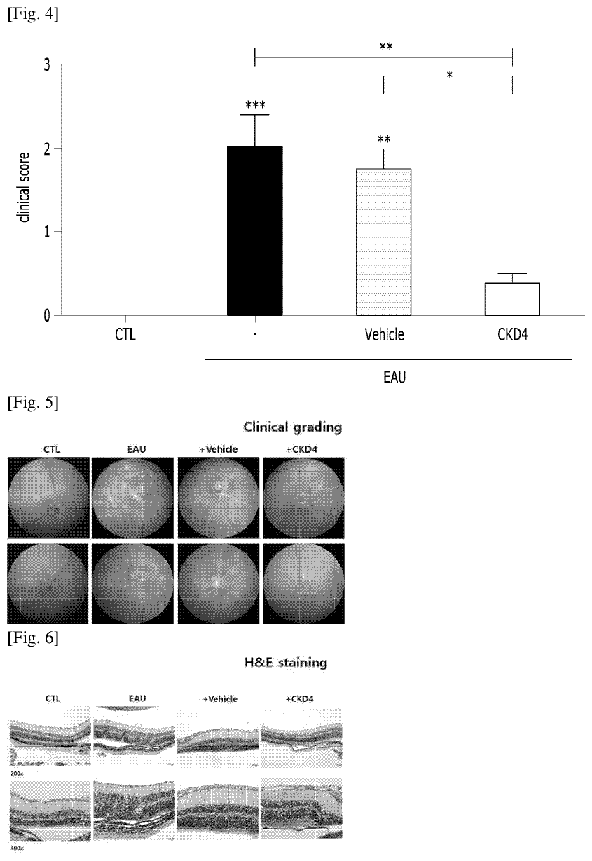

From the results, it was identified that a clinical grade in the EAU group rose near up to Grade 2, but the clinical grade in the EAU+CKD4 group (eye drop administration group) was significantly improved with Grade 0.5 or lower (FIGS. 4 and 5). The said experimental results show that the administration of the inventive compound by eye drop is effective in treating uveitis.

### Example 5. Identification of an Improvement Effect on Histopathological Lesions in the EAU Mouse

To see a degree of inflammation upon the occurrence of uveitis, eye balls, which were removed from mice on Day 21, were prepared into a paraffin block, and then hematoxylin & eosin (H&E) staining was performed thereon in a conventional manner.

From the results, a thick infiltration of inflammatory cells, granulomatous lesions, and inflammatory lesions such as edema and wrinkle were observed in retina tissues of the EAU group, but it was also identified that such inflammatory lesions were significantly decreased in the EAU+CKD4 group (eye drop administration group) (FIG. 6). The said experimental results show that the administration of the inventive compound by eye drop effectively removes an inflammation of an eye ball, to which uveitis occurs.

### Example 6. Identification of Results of Immunofluorescent Staining on Immunomarker in the EAU Mouse

To see whether immune cells infiltrate into an inflammatory region or not upon the occurrence of uveitis, immunofluorescent staining was performed on the paraffin block prepared in Example 2, targeting CD3, CD4, B220 (Abcam), HDAC6 (cell signaling) and Ace α-tubulin (Sigma), and then pictures thereof were taken by means of a confocal microscope (LSM780, Zeiss).

From the results, it was identified that a CD3(+) cell, which is a T cell marker, was considerably increased in the EAU group, but was significantly decreased to a similar level to that of a normal mouse in the EAU+CKD4 group (eye drop administration group) (FIG. 7). On the other hand, it was found that B220, which is a B cell marker, also showed a sign of being positive in the EAU group, but did not show an obvious change in the EAU+CKD4 group (eye drop administration group) (FIG. 7).

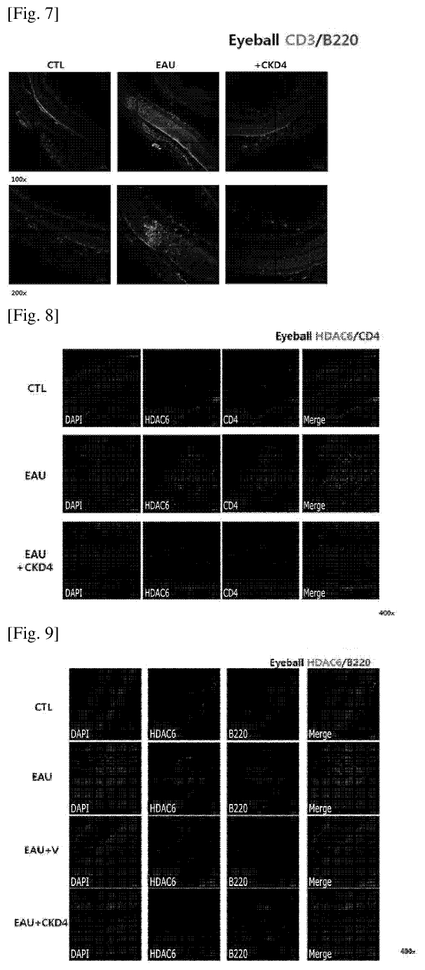

Also, HDAC6 showed a high degree of agreement with CD4. It was identified that the infiltration of CD4(+) immune cells into a retina tissue was increased in the EAU group, but was clearly decreased and remained only under the retina in the EAU+CKD4 group (eye drop administration group) (FIG. 8). In the meantime, the results of immunofluorescent staining on B220 and α-tubulin were observed as a quite different pattern from HDAC6 (FIGS. 9 and 10).

The experimental results above show that the administration of the inventive compound by eye drop effectively inhibits the infiltration of immune cells into an inflammatory region of uveitis.

### Example 7. Identification of a Decrease in a Level of Inflammatory Cytokines Through Enzyme-Linked Immunosobent Assay (ELISA) in the EAU Mouse

To obtain a spleen mononuclear cell (MNC), an EAU mouse spleen was crushed by means of a cell strainer, and then the MNC was obtained by means of HISTOPAQUE 1083. 5×10 5 MNCs were seeded into a flat-bottomed microtiter plate (96 well) with 200 μl/well in an RPMI 1640 medium without an addition of fetal bovine serum (FBS). Each well was stimulated with IRBP peptide at a concentration of 30 g/m, and subjected to reaction at 37° C., 5% CO₂ for 72 hours. After that, a supernatant fluid was obtained and analyzed by using an IFN-γ, IL-17 (Biolegend) ELISA set.

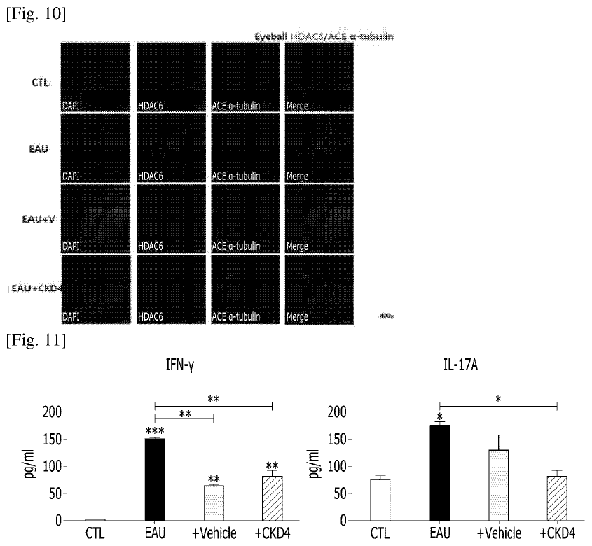

From the results, it was identified that the levels of both INF-γ and IL-17A were considerably increased in the EAU group in comparison with a normal mouse, but such levels of INF-γ and IL-17A were significantly decreased in the EAU+CKD4 group (eye drop administration group) (FIG. 11). The said experimental results show that the administration of the inventive compound by eye drop effectively decreases a level of systemic inflammatory cytokines in uveitis.

### Example 8. Identification of a Decrease in the Level of Inflammatory Cytokines Through a Real-Time PCR in the EAU Mouse

To perform a real-time PCR, a retina was first isolated from an eye ball, which had been removed from the EAU mouse, and then cells thereof were dissolved in a Trizol reagent. Then, cDNA was synthesized with regard to an RNA sample by using PrimeScript RT Master (TAKARA). After that, the real-time PCR was performed with StepOnePlusReal-Time PCR System (Applied Biosystems), using SYBR Premix Ex Tap (TAKARA) and a primer specific to each of the genes (IL-1p, TNF-α, IFN-γ, IL-17, HDAC6). The base sequences of the primer used in an experiment are as follows (Table 3).

TABLE 3

| Target Gene | Base Sequence | |
|---|---|---|
| mHDAC6 | (F) | 5′-AAGTGGAAGAAGCCGTGCTA-3′ |
| | | (SEQ ID NO: 1) |
| | (R) | 5′-CTCCAGGTGACACATGATGC-3′ |
| | | (SEQ ID NO: 2) |
| mIL-17 | (F) | 5′-ACCAGCATCTTCTCGACCCTGAAA-3′ |
| | | (SEQ ID NO: 3) |
| | (R) | 5′-ACCAGCATCTTCTCGACCCTGAAA-3′ |
| | | (SEQ ID NO: 4) |
| mIFN-γ | (F) | 5′-ACAAAGATGGCAGAGCACGA-3′ |
| | | (SEQ ID NO: 5) |
| | (R) | 5′-TCCACCAACATGTGCGGTTT-3′ |
| | | (SEQ ID NO: 6) |
| mIL-1β | (F) | 5′-AAGGGCTGCTTCCAAACCTTTGAC-3′ |
| | | (SEQ ID NO: 7) |
| | (R) | 5′-ATACTGCCTGCCTGAAGCTCTTGT-3′ |
| | | (SEQ ID NO: 8) |
| mIL-6 | (F) | 5′-TGGCTAAGGACCAAGACCAT-3′ |
| | | (SEQ ID NO: 9) |
| | (R) | 5′-TAACGCACTAGGTTTGCCGA-3′ |
| | | (SEQ ID NO: 10) |
| TNF-α | (F) | 5′-AGCCGATGGGTTGTACCTTGTCTA-3′ |
| | | (SEQ ID NO: 11) |
| | (R) | 5′-TGAGATAGCAAATCGGCTGACGGT-3′ |
| | | (SEQ ID NO: 12) |
| β-actin | (F) | 5′-AGGGAAATCGTGCGTGACAT-3′ |
| | | (SEQ ID NO: 13) |
| | (R) | 5′-AACCGCTCGTTGCCAATAGT-3′ |
| | | (SEQ ID NO: 14) |

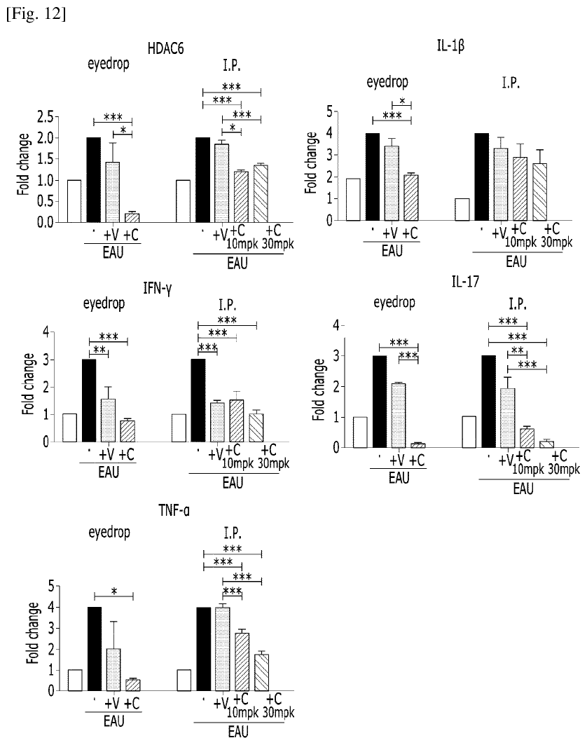

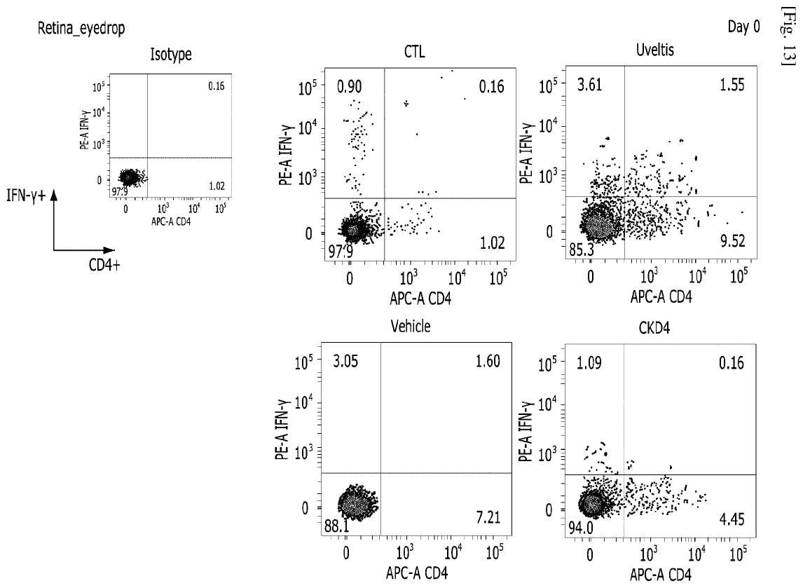

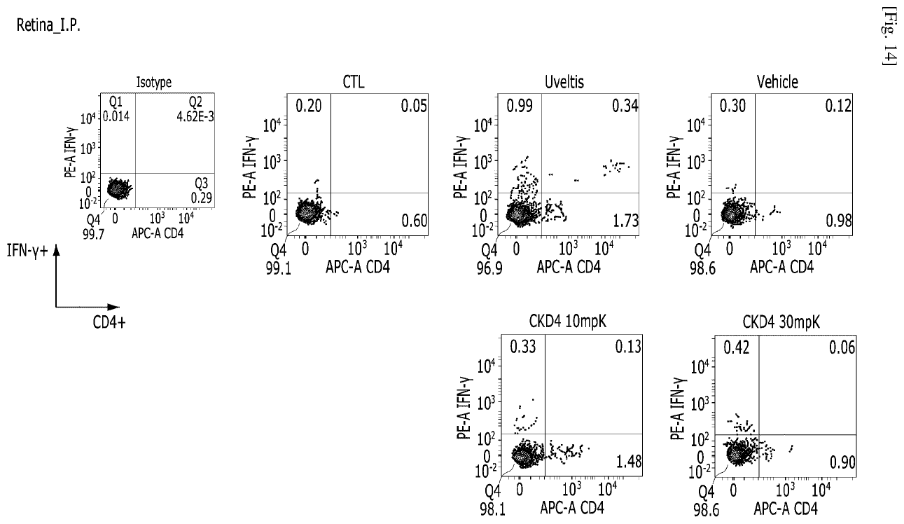

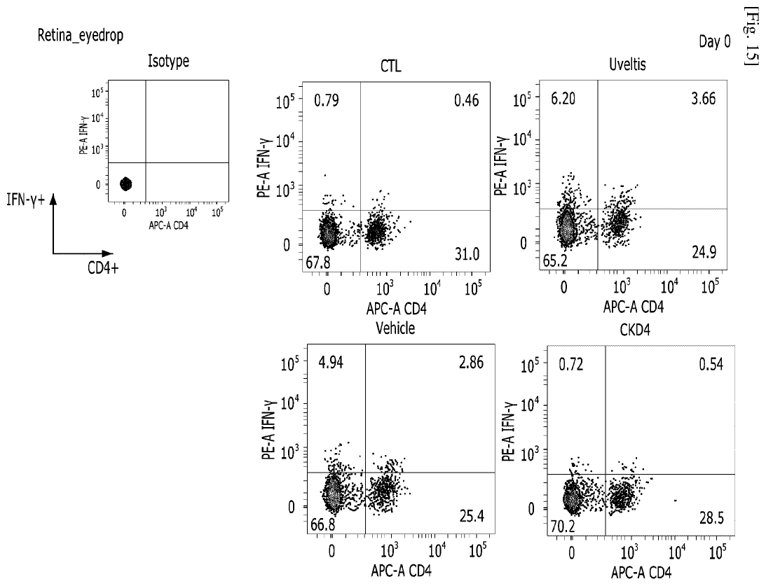

From the results, it was identified that an HDAC6 value was increased in the EAU group, and the levels of IL-1β, INF-γ, IL-17 and TNF-α, which are inflammatory factors, were considerably increased, too. In the EAU+CKD4 group, however, it was identified that the expression levels of HDAC6 and the said inflammatory factors were significantly decreased and recovered to the level of the normal mouse in both eye drop and i.p. administration groups (FIG. 12). The said experimental results show that the administration of the inventive compound by eye drop effectively decreases the level of inflammatory cytokines in an inflammatory region of uveitis.

### Example 9. Observation of Changes in Immune Cells Through a Flow Cytometer (FACS) in the EAU Mouse

Changes in the expression of cytokines as well as immune cells infiltrating upon the occurrence of uveitis were identified in a retina or a draining lymph node by means of a flow cytometer (fluorescence activated cell sorter, FACS).

Particularly, the retina or draining lymph node tissue was made into a single cell, then CD4, CD19, CD45, CD11b, F4/80 (Biolegend), etc. which are cell surface markers, were stained, and then IFN-γ, IL-17 and IL-1β (Biolegend), which are intra-cellular markers, were stained by fixing the cells and performing permeabilization. Then, an expression pattern of each marker was identified by means of the flow cytometer (Flow cytometry, Canto II).

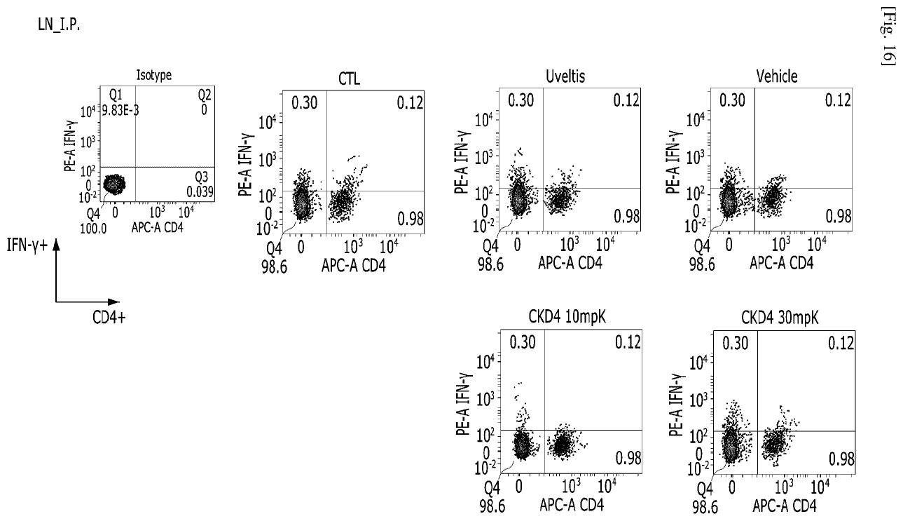

From the results, it was identified that the number of INF-γ(+)/CD4(+) cells was increased in the retina tissues of mice in the EAU group, but was significantly decreased in the eye drop administration group (FIG. 13) and the i.p. administration group (FIG. 14; corresponding to both 10 and 30 mg/kg) in case of the EAU+CKD4 group (FIGS. 13 and 14). Likewise, it was identified that the number of INF-γ(+)/CD4(+) cells was also increased in the lymph node tissues of mice in the EAU group, but the number of the said cells was significantly decreased in both eye drop administration group (FIG. 15) and i.p. administration group (FIG. 16) in case of the EAU+CKD4 group (FIGS. 15 and 16).

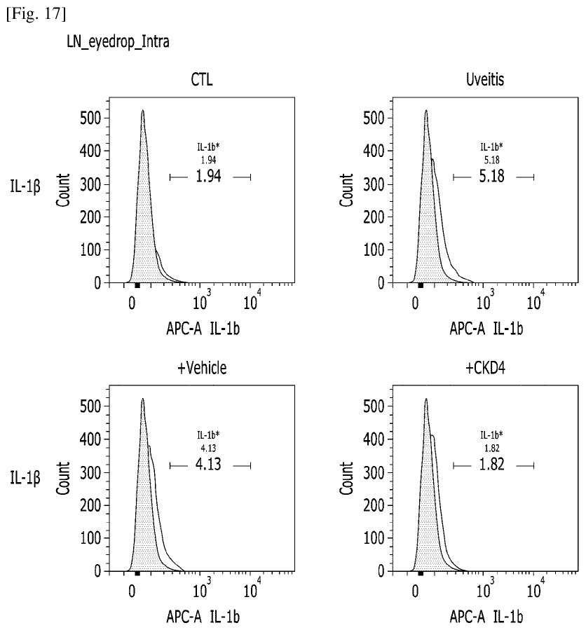

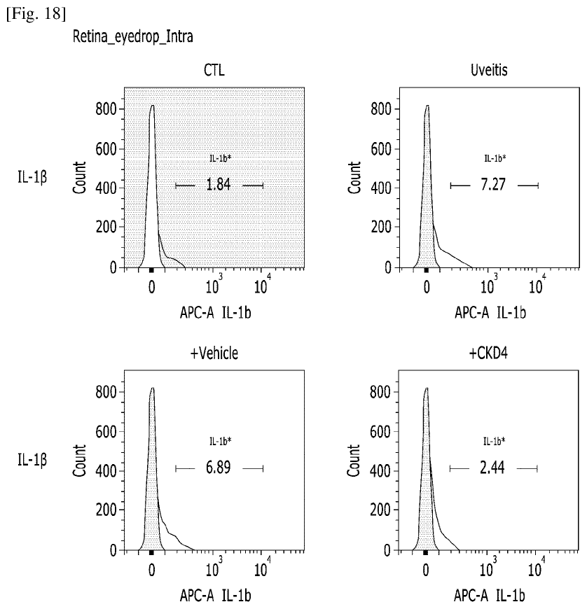

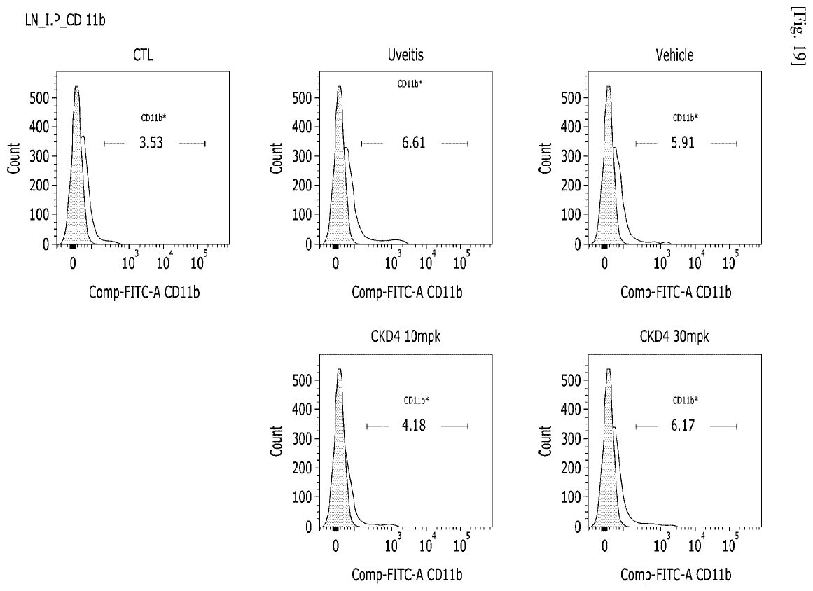

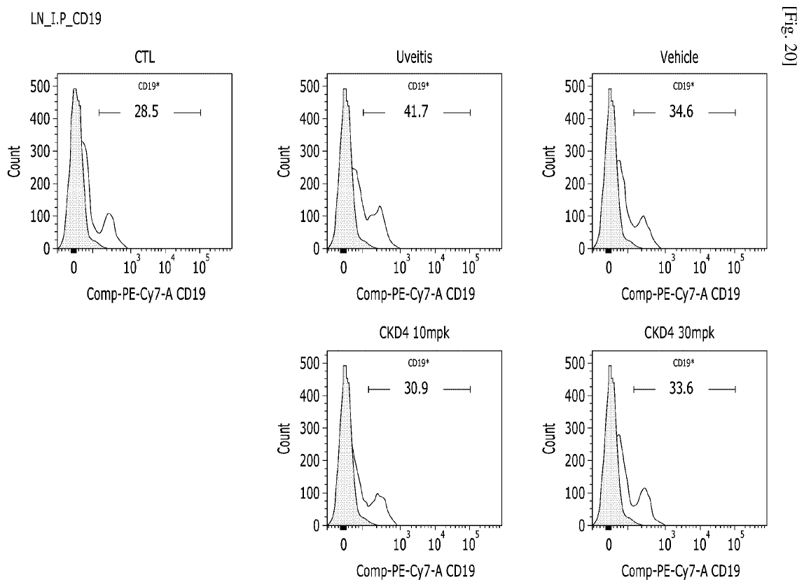

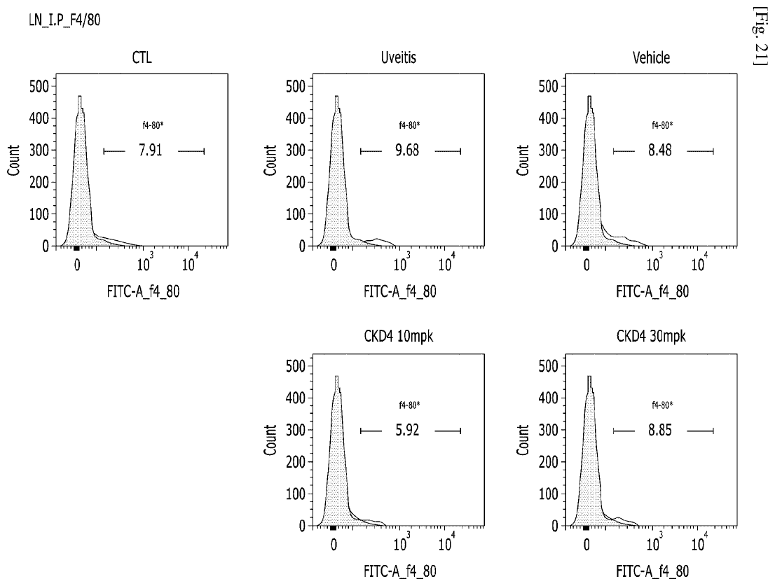

On the other hand, it was identified that the number of IL-IP(+) cells was considerably increased in both retina and lymph node tissues of mice in the EAU group, but was significantly decreased in the eye drop administration group in case of the EAU+CKD4 administration group (FIGS. 17 and 18). Also, it was identified that each number of CD11b(+), CD19(+) and F4/80(+) cells was also considerably increased in the lymph nodes of mice in the EAU group, but was decreased in the EAU+CKD4 administration group (i.p. administration group) (FIGS. 19 to 21).

The experimental results above show that the compound according to the present invention achieves an excellent immunosuppressant effect on uveitis, such that it may act as an effective therapeutic agent for uveitis.

While specific portions of the present invention have been described in detail above, it is apparent to those skilled in the art that such detailed descriptions are set forth to illustrate preferred exemplary embodiments only, but not construed to limit the scope of the present invention. Thus, it should be understood that the substantial scope of the present invention is defined by the accompanying claims and equivalents thereto.

1. A pharmaceutical composition for preventing or treating uveitis, comprising a compound represented by a following formula I, an optical isomer thereof or a pharmaceutically acceptable salt thereof as an effective component: 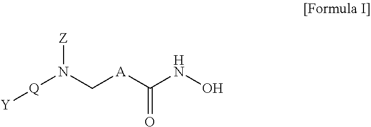
wherein
A is 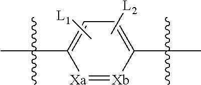
Xa and Xb are each independently CH or N,
L₁ and L₂ are each independently hydrogen, halogen, —CF₃ or —C₁₋₃ straight or branched chain alkyl,
Q is C(═O), S(═O)₂, S(═O) or C(═NH),
Y is selected from a following group: 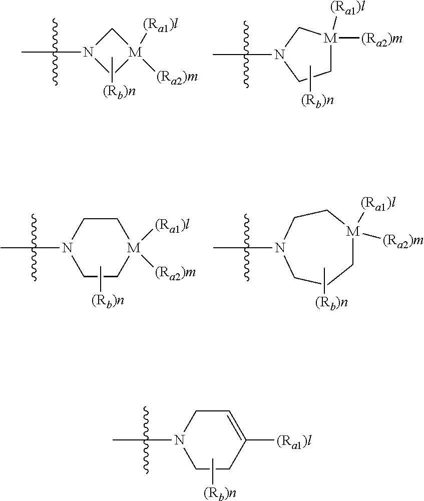
M is C, N, O, S or S(═O)₂, wherein, at this time, in case M is C, l and m are 1; in case M is N, l is 1 and m is 0; and in case M is 0, S or S(═O)₂, l and m are 0,
Rₐ₁ and Rₐ₂ are each independently hydrogen; hydroxy; —C₁₋₄ straight or branched chain alkyl, which is unsubstituted or substituted with at least one halogen; —C₁₋₄ straight or branched chain alcohol; benzhydryl; —C₁₋₄ straight or branched chain alkyl, which is substituted with a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; a saturated or unsaturated 5- to 7-membered heterocyclized compound comprising 1 to 3 heteroatoms of N, O or S as a ring member, wherein, at this time, the heterocyclized compound may be unsubstituted or at least one hydrogen may be optionally substituted with OH, OCH₃, CH₃, CH₂CH₃ or halogen; phenyl, wherein it is unsubstituted or at least one hydrogen is substituted with halogen, C₁₋₄ alkoxy, C₁₋₂ alkyl or hydroxy; benzyl, wherein it is unsubstituted or at least one hydrogen is substituted with halogen, C₁₋₄ alkoxy, C₁₋₂ alkyl or hydroxy; —S(═O)₂CH₃; halogen; —C₁₋₆ straight or branched chain alkoxy; —C₂₋₆ alkoxyalkyl; —C(═O)Rₓ, wherein Rₓ is straight or branched chain C₁₋₃ alkyl or C₃₋₁₀ cycloalkyl; 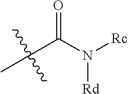
wherein R_{c} and R_{d} are each independently hydrogen, C₁₋₃ straight or branched chain alkyl; and 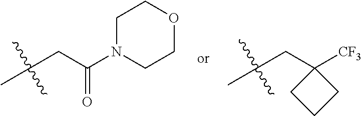 [SMILES: *CC(=O)N1CCOCC1 ; *CC1(C(F)(F)F)CCC1]
n is an integer of 0, 1 or 2,
R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or 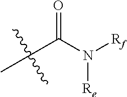
Rₑ and R_{f} are each independently hydrogen or —C₁₋₃ straight or branched chain alkyl,
Z is selected from a following group: 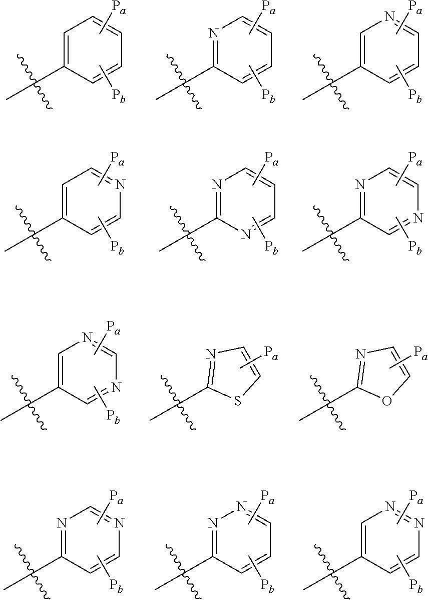
Pₐ and P_{b} are each independently 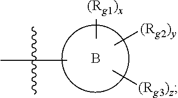
hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆straight or branched chain alkyl alkoxy; —CH₂F; or —C₁₋₃ alcohol,
here 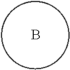
is phenyl, pyridine, pyrimidine, thiazole, indole, indazole, piperazine, quinoline, furan, tetrahydropyridine, piperidine or a ring selected from a following group: 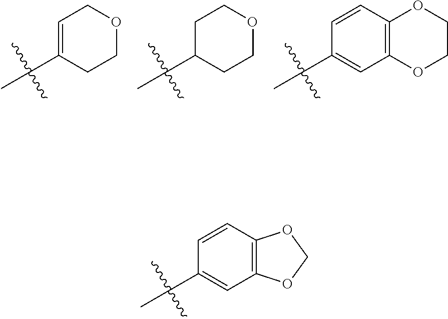 [SMILES: *C1=CCOCC1 ; *C1CCOCC1 ; *c1ccc2c(c1)OCCO2 ; *c1ccc2c(c1)OCO2]
x, y and z are each independently an integer of 0 or 1,
R_{g1}, R_{g2} and R_{g3} are each independently hydrogen; hydroxy; —C₁₋₃ alkyl; —CF₃; —C₁₋₆ straight or branched chain alkoxy; —C₂₋₆ straight or branched chain alkyl alkoxy; —C(═O)CH₃; —C₁₋₄ straight or branched chain hydroxyalkyl; —N(CH₃)₂; halogen; phenyl; —S((═O)₂)CH₃; or selected from a following group:

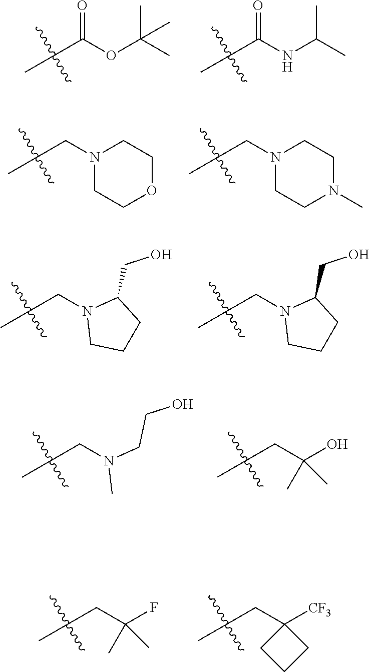

2. The pharmaceutical composition, according to claim 1, wherein the compound represented by the formula I above is a compound represented by a following formula Ia: 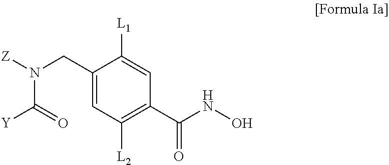
wherein
L₁ and L₂ are each independently hydrogen or halogen,
Y is 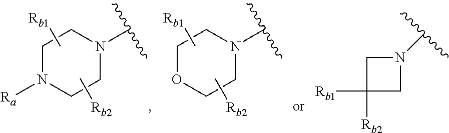
Z is phenyl or pyridinyl wherein, at least one hydrogen of phenyl or pyridinyl may be substituted with halogen, CF₃ or CF₂H.

3. The pharmaceutical composition, according to claim 2, wherein the compound represented by the formula Ia above is a compound described in a following table:

| Compound | Structure |
|---|---|
| 255 | 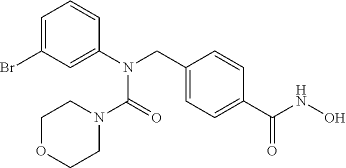 [SMILES: O=C(NO)c1ccc(CN(C(=O)N2CCOCC2)c2cccc(Br)c2)cc1] |
| 280 | 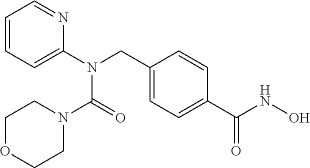 [SMILES: O=C(NO)c1ccc(CN(C(=O)N2CCOCC2)c2ccccn2)cc1] |
| 374 | 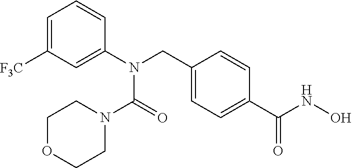 [SMILES: O=C(NO)c1ccc(CN(C(=O)N2CCOCC2)c2cccc(C(F)(F)F)c2)cc1] |
| 416 | 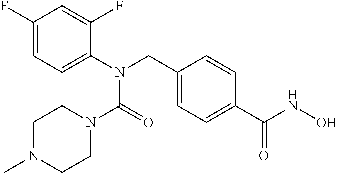 [SMILES: CN1CCN(C(=O)N(Cc2ccc(C(=O)NO)cc2)c2ccc(F)cc2F)CC1] |
| 461 | 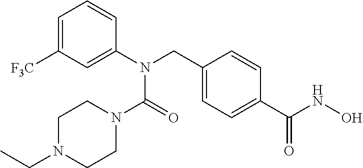 [SMILES: CCN1CCN(C(=O)N(Cc2ccc(C(=O)NO)cc2)c2cccc(C(F)(F)F)c2)CC1] |
| 476 | 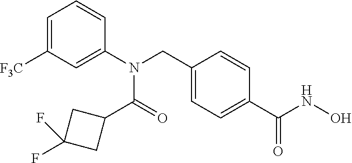 [SMILES: O=C(NO)c1ccc(CN(C(=O)C2CC(F)(F)C2)c2cccc(C(F)(F)F)c2)cc1] |
| 500 | 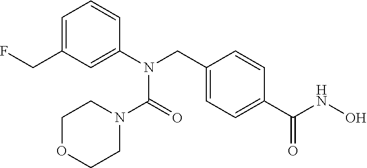 [SMILES: O=C(NO)c1ccc(CN(C(=O)N2CCOCC2)c2cccc(CF)c2)cc1] |
| 530 | 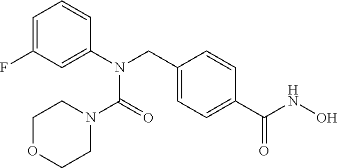 [SMILES: O=C(NO)c1ccc(CN(C(=O)N2CCOCC2)c2cccc(F)c2)cc1] |
| 532 | 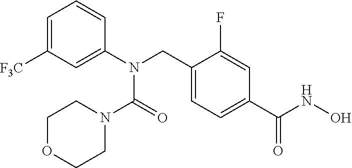 [SMILES: O=C(NO)c1ccc(CN(C(=O)N2CCOCC2)c2cccc(C(F)(F)F)c2)c(F)c1] |

4. The pharmaceutical composition, according to claim 1, wherein the said uveitis is anterior uveitis, intermediate uveitis, posterior uveitis or pan-uveitis.

5. The pharmaceutical composition, according to claim 1, wherein the said uveitis is infectious uveitis or non-infectious uveitis.

6. The pharmaceutical composition, according to claim 1, wherein the said pharmaceutical composition is administered parenterally.

7. The pharmaceutical composition, according to claim 1, wherein the said pharmaceutical composition is administered by eye drop.

8. The pharmaceutical composition, according to claim 1, wherein the said pharmaceutical composition is administered intraperitoneally.

9. A method for treating uveitis, comprising administering a therapeutically effective amount of the compound represented by formula I, an optical isomer thereof or a pharmaceutically acceptable salt thereof according to claim 1.

10. Use of the compound represented by formula I, an optical isomer thereof or a pharmaceutically acceptable salt thereof according to claim 1, in the manufacture of a medicament for treating uveitis.
